(19) United States
(12) Patent Application Publication
(10) Pub. No.: US 2016/0249840 A1
(43) Pub. Date: Sep. 1, 2016

(54) PH MICROSENSOR FOR GLUCOSE AND OTHER ANALYTE SENSOR FAULT DETECTION

(71) Applicant: Medtronic MiniMed, Inc., Northridge, CA (US)

(72) Inventors: Daniel E. Pesantez, Canoga Park, CA (US); Jia Yao, Reseda, CA (US); Bradley C. Liang, Bloomfield Hills, MI (US); Rajiv Shah, Rancho Palos Verdes, CA (US)

(21) Appl. No.: 14/632,731

(22) Filed: Feb. 26, 2015

(51) Int. Cl. A61B 5/1486; C12Q 1/00; A61B 5/145; G01N 27/327

(52) U.S. Cl. CPC A61B 5/14865; G01N 27/3272; G01N 27/3273; G01N 27/3274; C12Q 1/005; C12Q 1/006; A61B 5/14539; A61B 5/14532

(57) ABSTRACT

Embodiments of the invention provide amperometric analyte sensor systems comprising a plurality of electrodes including one or more electrodes designed to monitor pH in order to facilitate the sensing of analytes at different pH levels within a sensor environment. Typical embodiments of the invention include glucose oxidase based amperometric sensors used in the management of diabetes.

### TECHNICAL FIELD

The present invention relates to methods, materials and elements useful for analyte sensor systems, such as glucose sensors used in the management of diabetes.

### BACKGROUND OF THE INVENTION

Sensors are used to monitor a wide variety of compounds in various environments, including in vivo analytes. The quantitative determination of analytes in humans and mammals is of great importance in the diagnoses and maintenance of a number of pathological conditions. Illustrative analytes that are commonly monitored in a large number of individuals include glucose, lactate, cholesterol, and bilirubin. The determination of glucose concentrations in body fluids is of particular importance to diabetic individuals, individuals who must frequently check glucose levels in their body fluids to regulate the glucose intake in their diets. The results of such tests can be crucial in determining what, if any, insulin and/or other medication need to be administered.

Analyte sensors typically include components that convert interactions with analytes into detectable signals that can be correlated with the concentrations of the analyte. For example, some glucose sensors use amperometric means to monitor glucose in vivo. Some amperometric glucose sensors incorporate electrodes coated with layers of materials such as glucose oxidase (GOx), an enzyme that catalyzes the reaction between glucose and oxygen to yield gluconic acid and hydrogen peroxide (H₂O₂). The H₂O₂ formed in this reaction alters an electrode current to form a detectable and measurable signal. Based on the signal, the concentration of glucose in the individual can then be measured. A typical glucose sensor works according to the following chemical reactions:

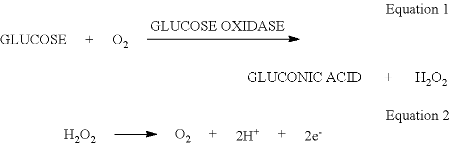

The glucose oxidase is used to catalyze the reaction between glucose and oxygen to yield gluconic acid and hydrogen peroxide as shown in equation 1. The H₂O₂ reacts electrochemically as shown in equation 2, and the current is measured by a potentiostat. The stoichiometry of the reaction provides challenges to developing in vivo sensors. In particular, for optimal sensor performance, sensor signal output should be determined only by the analyte of interest (glucose), and not by any co-substrates (O₂) or kinetically controlled parameters such as diffusion. If oxygen and glucose are present in equimolar concentrations, then the H₂O₂ is stoichiometrically related to the amount of glucose that reacts at the enzyme; and the associated current that generates the sensor signal is proportional to the amount of glucose that reacts with the enzyme. If, however, there is insufficient oxygen for all of the glucose to react with the enzyme, then the current will be proportional to the oxygen concentration, not the glucose concentration. Certain sensor designs address this oxygen deficit problem by using a series of layered materials selected to have specific function properties, for example an ability to selectively modulate the diffusion of oxygen and/or analytes. Problems associated with such designs can include, for example, pH changes within the layered sensor environment that, over time, can compromise the accuracy of sensor reading. Methods and elements designed to address such challenges in this technology are desirable.

### SUMMARY OF THE INVENTION

As noted above, amperometric glucose sensors commonly utilize electrodes coated with glucose oxidase (GOx), an enzyme that catalyzes the reaction between glucose and oxygen to yield gluconic acid and hydrogen peroxide (H₂O₂). The H₂O₂ formed in this reaction alters an electrode current to form a detectable analyte signal that is measured by the sensor. Based on such signals, the concentration of glucose can then be measured. As disclosed herein, acidic compounds such as gluconic acid that are also produced by enzymes such as glucose oxidase can alter the pH of the sensor in a manner that can alter the current readings that are used to observe analyte. Embodiments of the invention include sensor systems designed to address this phenomena by including an electrode designed to monitor pH within the sensor system. In such systems, the pH is monitored so that analyte signals are calculated in a pH specific manner in order to increase sensor accuracy and sensitivity.

The invention disclosed herein has a number of embodiments including amperometric analyte sensor systems and methods for making and using them. Such systems can comprise, for example, a plurality of electrodes disposed on a base including a working electrode, a counter electrode, a reference electrode, and a pH electrode that is designed to be responsive to changes in pH within the sensor system. Such systems further include a processor and a computer-readable program having instructions which cause the processor to assess signal data obtained from the working electrode and the pH electrode. In these systems the working electrode and the processor are coupled so that the working electrode monitors analyte within the sensor system, and the pH electrode and the processor are coupled so that the pH electrode monitors pH within the sensor system. In order to take into account the effects of pH on current measured at the working electrode, the processor uses a first algorithm to calculate a concentration of analyte when the pH of the sensor system is at or above pH 7.1, and then a second algorithm to calculate a concentration of analyte when the pH of the sensor system is below pH 6.9. Typically the first and second algorithms are designed to factor in how different pH can modulate amperometric current observed at the working electrode in the presence of analyte. For example, in certain embodiments of the invention, the second algorithm calculates the concentration of analyte considering an at least 10% drop in analyte signal that results from the pH of the sensor system changing from at or above pH 7.1 to below pH 6.9.

In typical embodiments of the invention, the working electrode of the amperometric analyte sensor system is coated with a plurality of layered materials including an analyte sensing layer comprising an oxidoreductase that produces hydrogen peroxide and an acidic compound in the presence of analyte (e.g. glucose oxidase, lactate oxidase and the like). The electrodes in the sensor can also be coated with other layers such as an interference rejection layer, a protein layer, an adhesion promoting layer and/or an analyte modulating layer comprising a composition that modulates the diffusion of an analyte diffusing therethrough. The electrodes in such systems can be formed from a wide variety of materials. In some embodiments of the invention, the pH electrode and/or the working electrode comprises iridium oxide. In some embodiments of the invention, the pH electrode also functions as the working electrode. In some embodiments of the invention, the working electrode comprises platinum black coated with a glucose oxidase composition that forms gluconic acid and hydrogen peroxide in the presence of glucose. In embodiments of the invention, the pH electrode and the working electrode can both be in operable contact with the reference electrode and the counter electrode. In certain embodiments, the pH electrode continuously monitors the open circuit potential between the pH electrode and the reference electrode.

A related embodiment of the invention is a method of calculating the concentration of glucose at a plurality of different pH values within an amperometric glucose sensor. Typically this method comprises placing an amperometric glucose sensor into an environment comprising glucose. In such embodiments, the amperometric analyte sensor is part of a system comprising a plurality of electrodes disposed on a base including a working electrode coated with an analyte sensing layer comprising glucose oxidase that produces gluconic acid and hydrogen peroxide in the presence of glucose; and also an analyte modulating layer, wherein the analyte modulating layer comprises a composition that modulates the diffusion of an analyte diffusing through the analyte modulating layer. This glucose sensing system includes a counter electrode, a reference electrode and a pH electrode designed to observe changes in pH within the local sensor system environment. This glucose sensing system also includes a processor and a computer-readable program having instructions which cause the processor to assess signal data obtained from the working electrode and the pH electrode.

In this amperometric glucose sensor system, the working electrode and the processor are coupled so that the working electrode monitors glucose within the sensor system, and the pH electrode and the processor are coupled so that the pH electrode monitors the pH of the sensor within the sensor system. In the method for calculating the concentration of glucose at a plurality of different pH values within an amperometric glucose sensor one then monitors the pH of the sensor within the sensor system in combination with monitoring glucose within the sensor system; and then calculating the concentration of glucose. In the system, the processor uses a first set of parameters to calculate a concentration of glucose when the pH of the analyte sensing layer is at or above pH 7.1; and the processor uses a second set of parameters to calculate a concentration of glucose when the pH of the analyte sensing layer is below pH 6.9. Optionally, for example, the second set of parameters calculates the concentration of analyte using an at least 10% drop in analyte signal that results from the pH of the sensor system changing from at or above pH 7.1 to below pH 6.9.

In certain embodiments of the invention, the pH electrode continuously monitors the open circuit potential between the pH electrode and the reference electrode. In some of these embodiments of the invention, the system switches from using the first set of parameters to using the second set of parameters when the open circuit potential is above or below a predefined value that is between 20 millivolts and 180 millivolts. Optionally the method includes calculating glucose concentrations using a calibration curve of the relationship between current and pH at the working electrode within the sensor.

Yet another embodiment of the invention is a method of making an analyte sensor comprising the steps of providing a base layer and then forming a conductive layer on the base layer, wherein the conductive layer includes a plurality of electrodes including a pH electrode, a working electrode, a reference electrode and a counter electrode. The method further comprises forming an analyte sensing layer over the working electrode, wherein the analyte sensing layer comprises a polypeptide that forms an acidic compound in the presence of the analyte; and then forming an analyte modulating layer disposed over the analyte sensing layer, wherein the analyte modulating layer includes a composition that modulates the diffusion of the analyte therethrough. Optionally the method includes forming an adhesion promoting layer on the analyte sensing layer or a protein layer. Some embodiments of the invention include forming a cover layer disposed on at least a portion of the analyte modulating layer, wherein the cover layer further includes an aperture over at least a portion of the analyte modulating layer.

Typically this analyte sensor apparatus is then operably coupled to a process comprising a computer-readable program having instructions which cause the processor to assess signal data obtained from the working electrode and the pH electrode. In such embodiments, the working and/or pH electrode and the processor are coupled so that the working electrode monitors analyte within the sensor system and the pH electrode monitors pH of the sensor within the sensor system; and the processor uses a first algorithm to calculate a concentration of analyte when the pH of the sensor system is at or above pH 7.1; and the processor uses a second algorithm to calculate a concentration of analyte when the pH of the sensor system is below pH 6.9.

Yet another embodiment of the invention is a method of sensing an analyte within the body of a mammal. Typically this method comprises implanting an analyte sensor as disclosed herein within the mammal (e.g. in the interstitial space of a diabetic individual), sensing an alteration in current at the working electrode in the presence of the analyte while simultaneously sensing pH within the sensor system; and then correlating the alteration in current at the observed pH with the presence of the analyte, so that the analyte is sensed. While typical embodiments of the invention pertain to glucose sensors, the layered compositions disclosed herein can be adapted for use with a wide variety of devices known in the art.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1A provides a diagram of different electrode configurations for working, pH, counter and reference electrodes. FIG. 1B shows a schematic of a sensor design comprising an amperometric analyte sensor formed from a plurality of planar layered elements.

FIG. 2 provides a perspective view illustrating a subcutaneous sensor insertion set, a telemetered characteristic monitor transmitter device, and a data receiving device embodying features of the invention.

FIG. 3 shows a schematic of chemical reactions that decrease pH in an amperometric glucose sensor formed from a plurality of planar layered elements. In particular, during glucose sensor in vivo operation, the accumulation of enzymatically generated products (e.g. gluconic acid) can lower the local pH within the sensor chemistry layers. The decrease in pH levels can negatively impact sensor signal and possibly contribute to sensitivity loss observed on sensors in vivo, adding to bio-fouling and immune response effects.

FIGS. 4A-4G provide graphs of data showing that readings in amperometric GOx based glucose sensors are impacted by pH change. FIG. 4A provides a graph of data showing how increasing concentrations of gluconic acid decreases solution pH level. FIG. 4B provides a graph of data showing how GOx enzyme activity decreases at non-neutral pH. FIG. 4C provides a graph of data showing how Glucose Limiting Membrane permeability decreased at pH<6. FIG. 4D provides a graph of data showing how sensor signal decreased with increasing concentration of Gluconic Acid. FIGS. 4E-4G provide graphs of data showing pH induced % of Isig change on various amperometric glucose sensor embodiments.

FIGS. 5A-5C provide graphs of data showing how IrOx electrode microsensors demonstrate high pH sensitivity. FIG. 5A provides a graph of data showing how IrOx electrodes demonstrate a linear response to pH with a sensitivity of 70 mV/pH. FIG. 5B provides a graph of data from a dynamic pH test which demonstrates IrOx electrode fast response. FIG. 5C provides a graph of data showing how localized pH changes can be induced by enzymatically generated H₂O₂ and gluconic acid.

FIGS. 6A-6B provide graphs of data showing how IrOx electrode microsensors demonstrate glucose sensing capability in standard glucose calibration tests. FIG. 6A provides a graph of data showing how IrOx working electrodes demonstrated linear current signal with glucose concentration change. FIG. 6B provides a graph of data showing that this linearity is is comparable to control sensor having a working electrode formed from platinum black.

FIGS. 7A-7C provide graphs of data showing how pH electrode microsensors can be used to correlate open circuit potential (OCP) with pH with a high degree of sensitivity and accuracy. In these studies, six different electrodes were tested over a period of 5 minutes in PBS buffers with 5 different pH values: 4, 6, 7.3, 8 and 10. In these studies, a very small drift was observed. The linearity for all the electrodes was good, all with an R2>95%. The sensitivity for the 6 sensors was from 51 to 74 mV/pH, avg=62 mV/pH.

FIGS. 8A, 8B and 8C show illustrative calibration curves that a glucose concentration determining algorithm could use by taking into account OCP VS. pH (FIG. 8A) and Isig VS. pH change (FIG. 8B). The plot in FIG. 8C shows Isig changes corresponding to pH changes. This plot shows data from solutions controlled to have a pH to five acidic levels and three alkaline levels. This data shows that % of Isig change increases as pH gets more acidic, while Isig remained relatively stable at alkaline levels. Such empirical data is useful for sensor specific algorithms. This data demonstrated the approximate Isig compensation range that can be used for sensor algorithms in correspondence to pH range where: (1) pH: 7.0-7.3 corresponds to an Isig % change˜7%; (2) pH: 6.75-7.0 corresponds to an Isig % change˜10%; (3) pH: 6.5-6.75 corresponds to an Isig % change˜12%; (4) pH: 6.25-6.5 corresponds to an Isig % change˜13%; (5) pH: 6.0-6.25 corresponds to an Isig % change˜14%; and (6) pH: 7.5-8.0 corresponds to an Isig % change˜3%. Such tests have shown that Isig can change up to 33% over a pH range from 6.34 to 7.41.

### DETAILED DESCRIPTION OF THE INVENTION

Unless otherwise defined, all terms of art, notations, and other scientific terms or terminology used herein are intended to have the meanings commonly understood by those of skill in the art to which this invention pertains. In some cases, terms with commonly understood meanings may be defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. A number of terms are defined below.

All numbers recited in the specification and associated claims that refer to values that can be numerically characterized with a value other than a whole number (e.g. a unit of measurement such as a concentration of a component in a composition) are understood to be modified by the term “about”. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Furthermore, all publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

The term “analyte” as used herein is a broad term and is used in its ordinary sense, including, without limitation, to refer to a substance or chemical constituent in a fluid such as a biological fluid (for example, blood, interstitial fluid, cerebral spinal fluid, lymph fluid or urine) that can be analyzed. Analytes can include naturally occurring substances, artificial substances, metabolites, and/or reaction products. In common embodiments, the analyte is glucose. However, embodiments of the invention can be used with sensors designed for detecting a wide variety other analytes. Illustrative analytes include but are not limited to, lactate as well as salts, sugars, proteins fats, vitamins and hormones that naturally occur in vivo (e.g. in blood or interstitial fluids). The analyte can be naturally present in the biological fluid or endogenous; for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin. The metabolic products of drugs and pharmaceutical compositions are also contemplated analytes.

The term “sensor” for example in “analyte sensor,” is used in its ordinary sense, including, without limitation, means used to detect a compound such as an analyte. A “sensor system” includes, for example, elements, structures and architectures (e.g. specific 3-dimensional constellations of molecular elements) designed to facilitate sensor use and function. Sensor systems can include, for example, compositions such as those having selected material properties, as well as electronic components such as elements and devices used in signal detection and analysis (e.g. current detectors, monitors, processors and the like).

As discussed in detail below, embodiments of the invention relate to the use of an electrochemical sensor that measures a concentration of an analyte of interest or a substance indicative of the concentration or presence of the analyte in fluid. In some embodiments, the sensor is a continuous device, for example a subcutaneous, transdermal, or intravascular device. In some embodiments, the device can analyze a plurality of intermittent blood samples. The sensor embodiments disclosed herein can use any known method, including invasive, minimally invasive, and non-invasive sensing techniques, to provide an output signal indicative of the concentration of the analyte of interest. Typically, the sensor is of the type that senses a product or reactant of an enzymatic reaction between an analyte and an enzyme in the presence of oxygen as a measure of the analyte in vivo or in vitro. Such sensors typically comprise a membrane surrounding the enzyme through which an analyte migrates. The product is then measured using electrochemical methods and thus the output of an electrode system functions as a measure of the analyte.

Embodiments of the invention disclosed herein provide sensors of the type used, for example, in subcutaneous or transcutaneous monitoring of blood glucose levels in a diabetic patient. A variety of implantable, electrochemical biosensors have been developed for the treatment of diabetes and other life-threatening diseases. Many existing sensor designs use some form of immobilized enzyme to achieve their bio-specificity. Embodiments of the invention described herein can be adapted and implemented with a wide variety of known electrochemical sensors, including for example, U.S. Patent Application No. 20050115832, U.S. Pat. Nos. 6,001,067, 6,702,857, 6,212,416, 6,119,028, 6,400,974, 6,595,919, 6,141,573, 6,122,536, 6,512,939 5,605,152, 4,431,004, 4,703,756, 6,514,718, 5,985,129, 5,390,691, 5,391,250, 5,482,473, 5,299,571, 5,568,806, 5,494,562, 6,120,676, 6,542,765, 7,033,336 as well as PCT International Publication Numbers WO 01/58348, WO 04/021877, WO 03/034902, WO 03/035117, WO 03/035891, WO 03/023388, WO 03/022128, WO 03/022352, WO 03/023708, WO 03/036255, WO03/036310 WO 08/042,625, and WO 03/074107, and European Patent Application EP 1153571, the contents of each of which are incorporated herein by reference.

### Illustrative Embodiments of the Invention and Associated Characteristics

Embodiments of the invention disclosed herein provide analyte sensors designed to include layered compositions that provide these sensors with enhanced functional and/or material properties. As discussed in detail below, typical embodiments of the invention relate to the use of a sensor that measures a concentration of an aqueous analyte of interest or a substance indicative of the concentration or presence of the analyte in vivo (e.g. glucose). In some embodiments of the invention, the sensor is a subcutaneous, intramuscular, intraperitoneal, intravascular or transdermal device. The sensor embodiments disclosed herein can use any known method, including invasive, minimally invasive, and non-invasive sensing techniques, to provide an output signal indicative of the concentration of the analyte of interest.

During in vivo operation of electrochemical glucose sensors, the accumulation of enzymatic generated products such as gluconic acid (GA) and hydrogen peroxide (H₂O₂) can lower the local pH within the sensor chemistry layers. This decrease of the local pH can contribute to sensitivity loss observed on sensors in vivo, adding to bio-fouling and immune response effects. Embodiments of the invention disclosed herein include a pH microsensor and sensor systems such as amperometric glucose sensors used in the management of diabetes as well as a new sensor fault detection method to optimized monitoring of analytes in such sensor systems. With such embodiments, micro-environmental effects can be proactively accounted by the proposed pH microsensor. Such a pH microsensor for glucose sensor fault detection/diagnostics would provide relevant feedback to a continuous glucose monitoring system to maintain glucose levels accurate, reliable and within an optimal range. In addition, sensor lifetime and duration of wear would be also extended, decreasing episodes of early sensor termination. Embodiments of the invention include micro-dual-sensors was built to detect glucose signal and the localized pH change within the sensor chemistry layers. In such embodiments, pH modulated signals can be evaluated using a smart diagnostics tool with algorithms to take corrective action and compensate for the pH mediated change, thus helping to sense glucose levels with a greater accuracy, reliability and sensitivity.

The invention disclosed herein has a number of embodiments including amperometric analyte sensor systems and methods for making and using them. Such systems can comprise, for example, a plurality of electrodes disposed on a base including a working electrode, a counter electrode, a reference electrode, and a pH electrode that is designed to be responsive to changes in pH within the sensor system. Such systems further include a processor and a computer-readable program having instructions which cause the processor to assess signal data obtained from the working electrode and the pH electrode. In these systems the working electrode and the processor are coupled so that the working electrode monitors analyte within the sensor system, and the pH electrode and the processor are coupled so that the pH electrode monitors pH within the sensor system. In order to take into account the effects of pH on current measured at the working electrode, the processor uses a first algorithm to calculate a concentration of analyte when the pH of the sensor system is at or above pH 7.1, and then a second algorithm to calculate a concentration of analyte when the pH of the sensor system is below pH 6.9. For example, in certain embodiments of the invention, the second algorithm calculates the concentration of analyte considering an at least 10% drop in analyte signal that results from the pH of the sensor system changing from at or above pH 7.1 to below pH 6.9. optionally the sensor is a glucose sensor and the processor uses a third algorithm/set of parameters to calculate a concentration of glucose when a pH range is between 6.75 and 7, wherein the third set of parameters includes calculations using pH mediated glucose signal change of at least 15% as compared to a same concentration of glucose at or above pH 7.3. Typically the first and second (and third etc.) algorithms are designed to factor in how different pH can modulate amperometric current observed at the working electrode in the presence of analyte. In certain embodiments, the sensor is a glucose sensor and the processor uses an algorithm/set of parameters to calculate a concentration of glucose when a pH range is between 6.5 and 6.75, 7 or 7.25 wherein the parameters includes calculations using pH mediated signal change of at least 10%, 15% or 20% as compared to a same concentration of glucose at or above pH 7.3.

In typical embodiments, the working electrode of the amperometric analyte sensor system is coated with a plurality of layered materials including an analyte sensing layer comprising an oxidoreductase that produces hydrogen peroxide and an acidic compound in the presence of analyte (e.g. glucose oxidase, lactate oxidase and the like). The electrodes in the sensor can also be coated with other layers such as an interference rejection layer, a protein layer, an adhesion promoting layer and/or an analyte modulating layer comprising a composition that modulates the diffusion of an analyte diffusing therethrough. The electrodes in such systems can be formed from a wide variety of materials and disposed in a variety of configurations. For example, the pH sensing electrode can be made of, but not limited to, the following materials or combination of materials: metals (e.g. platinum, palladium, ruthenium, osmium); metal oxides (e.g. iridium oxide, silver oxide, tin oxide); polymers and conducting polymers (e.g. ionophores, polypyrrole, polyaniline); and/or hydrogels (e.g. polyacrylic acid, chitosan with backbone functionalized with acidic or basic groups). —The electrodes can be arranged and configured in multiple ways: including single pH sensing electrode/stand alone or as a part of a sensing system; distributed along sensor probe; parallel to other analyte sensing electrodes; above or below other analyte sensing electrodes; on one or both sides of a 360 degree sensing probe; as part of a wire based electrode configuration and/or multiple and individually addressed pH electrodes on any of the above mentioned configurations.

A related embodiment of the invention is a method of calculating the concentration of glucose at a plurality of different pH values within an amperometric glucose sensor. Typically this method comprises placing an amperometric glucose sensor into an environment comprising glucose. In such embodiments, the amperometric analyte sensor is part of a system comprising a plurality of electrodes disposed on a base including a working electrode coated with an analyte sensing layer comprising glucose oxidase that produces gluconic acid and hydrogen peroxide in the presence of glucose; and also an analyte modulating layer, wherein the analyte modulating layer comprises a composition that modulates the diffusion of an analyte diffusing through the analyte modulating layer. This glucose sensing system includes a counter electrode, a reference electrode and a pH electrode designed to observe changes in pH within the local sensor system environment. This glucose sensing system also includes a processor and a computer-readable program having instructions which cause the processor to assess signal data obtained from the working electrode and the pH electrode. In this system, the working electrode and the processor are coupled so that the working electrode monitors glucose within the sensor system, and the pH electrode and the processor are coupled so that the pH electrode monitors the pH of the sensor within the sensor system. In the method for calculating the concentration of glucose at a plurality of different pH values within an amperometric glucose sensor one then monitors the pH of the sensor within the sensor system in combination with monitoring glucose within the sensor system; and then calculating the concentration of glucose. In the system, the processor uses a first set of parameters to calculate a concentration of glucose when the pH of the analyte sensing layer is at or above pH 7.1; and the processor uses a second set of parameters to calculate a concentration of glucose when the pH of the analyte sensing layer is below pH 6.9. Optionally, for example, the second set of parameters calculates the concentration of analyte using an at least 10% drop in analyte signal that results from the pH of the sensor system changing from at or above pH 7.1 to below pH 6.9.

As disclosed herein, embodiments of the invention include a pH microsensor designed to assess pH within the glucose sensor chemistry layers. Embodiments of the invention can comprise implanting an analyte sensor embodiment disclosed herein in to a mammal and then sensing one or more pH dependent electrical fluctuations such as alteration of open circuit potential (OCP) at the working electrode and correlating the alteration of OCP with pH levels, so that the local/internal pH is sensed. Implantation of the pH microsensor can take place into a variety of locations within the body of the mammal, for example in both vascular and non-vascular spaces. Embodiments of the invention utilize algorithms that compensate for pH dependent electrical fluctuations and adjust sensor sensitivity accordingly. In certain embodiments of the invention, the pH sensing electrode catalytic properties can be used to break down hydrogen peroxide and other electroactive species, therefore providing a superior platform to collect information on pH, glucose, lactate, potassium, calcium, oxygen, and/or any physiologically relevant analyte in the mammal.

In certain embodiments of the invention, the pH electrode continuously monitors the open circuit potential between the pH electrode and the reference electrode. In some of these embodiments of the invention, the system switches from using the first set of parameters to using the second set of parameters when the open circuit potential is above or below a predefined value that is between 20 millivolts and 180 millivolts. Optionally the method includes calculating glucose concentrations using a calibration curve of the relationship between current and pH at the working electrode within the sensor.

Yet another embodiment of the invention is a method of making an analyte sensor comprising the steps of providing a base layer and then forming a conductive layer on the base layer, wherein the conductive layer includes a plurality of electrodes including a pH electrode, a working electrode, a reference electrode and a counter electrode. The method further comprises forming an analyte sensing layer over the working electrode, wherein the analyte sensing layer comprises a polypeptide that forms an acidic compound in the presence of the analyte; and then forming an analyte modulating layer disposed over the analyte sensing layer, wherein the analyte modulating layer includes a composition that modulates the diffusion of the analyte therethrough. Optionally the method includes forming an adhesion promoting layer on the analyte sensing layer or a protein layer. Some embodiments of the invention include forming a cover layer disposed on at least a portion of the analyte modulating layer, wherein the cover layer further includes an aperture over at least a portion of the analyte modulating layer.

Typically this analyte sensor apparatus is then operably coupled to a process comprising a computer-readable program having instructions which cause the processor to assess signal data obtained from the working electrode and the pH electrode. In such embodiments, the working and/or pH electrode and the processor are coupled so that the working electrode monitors analyte within the sensor system and the pH electrode monitors pH of the sensor within the sensor system; and the processor uses a first algorithm to calculate a concentration of analyte when the pH of the sensor system is at or above a certain point, for example, pH 7.0, 7.1, 7.2 or 7.3; and the processor uses a second algorithm to calculate a concentration of analyte when the pH of the sensor system is below a certain point, for example, pH 7.0, 6.9, 6.8, 6.6 or 6.5.

Typically in such methods, the sensor is operably coupled to a processor comprising a computer-readable program having instructions which cause the processor to assess signal data obtained from the working electrode and the pH electrode. In such embodiments, the working electrode and the processor are coupled so that the working electrode monitors analyte within the sensor system, and the pH electrode and the processor are coupled so that the pH electrode monitors the pH of the sensor within the sensor system. In certain embodiments the processor uses a first algorithm to calculate a concentration of analyte when the pH of the sensor system is at or above a certain pH (e.g. pH 7.1); and the processor uses a second algorithm to calculate a concentration of analyte when the pH of the sensor system is below a certain pH (e.g. pH 6.9). Optionally the pH electrode is adapted to continuously monitor open circuit potential between the pH electrode and the reference electrode. In certain embodiments of the invention, the system switches from using the first algorithm to using a second algorithm when the open circuit potential is above or below a predefined value that is between 20 millivolts and 180 millivolts. In certain embodiments of the invention, the system switches from using a first algorithm to using a second algorithm when the open circuit potential reaches a predetermined threshold such as lower than 20 mV (˜pH=8) and/or higher than 180 mV

Embodiments of the invention use pH sensor reading to calibrate glucose sensor signal and achieve better accuracy in glucose sensor. In this context, there are a number of platforms where pH sensor reading can be used as a feedback to algorithm to compensate for pH mediated changes in glucose sensor signals. In typical embodiments of the invention, a pH sensor (e.g. one comprising an IrOx electrode) can be paired with a glucose sensor and the two can be placed very close to each other (e.g. less than 5, 4, 3, 2 or 1 millimeters apart) so that the pH changes on the pH sensor can be representative of the pH changes on the glucose sensor. In some embodiments, the pH sensor and glucose sensor can be fabricated on the same multi-electrode platform. Optionally, they can share the same Counter Electrode and Reference Electrode, but individual pH sensor and glucose sensor electrodes can function independently such that the glucose sensor can be operating in amperometric mode, and the pH sensor can be operating in Open Circuit Potential (OCP) mode. In some embodiments of the invention, the pH sensor can be operating in ON/OFF mode so that intervals of glucose sensing mode and pH sensing mode are continuously applied to the same pH sensor electrode. The OCP reading can then be used for an analysis of glucose sensing signal.

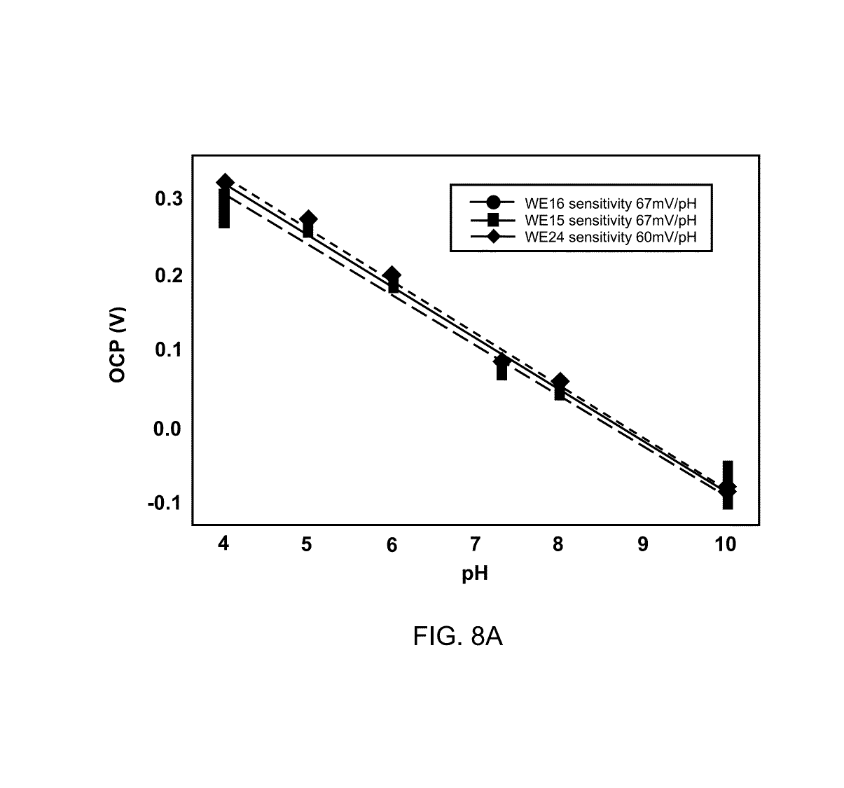

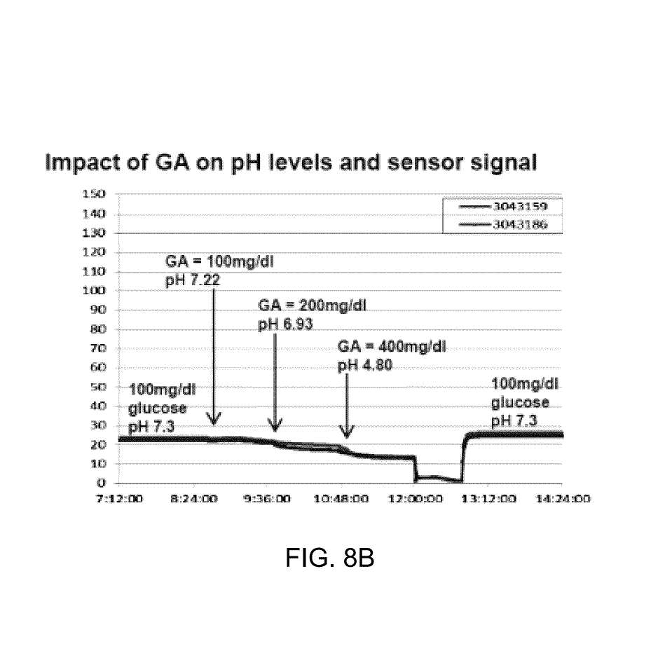

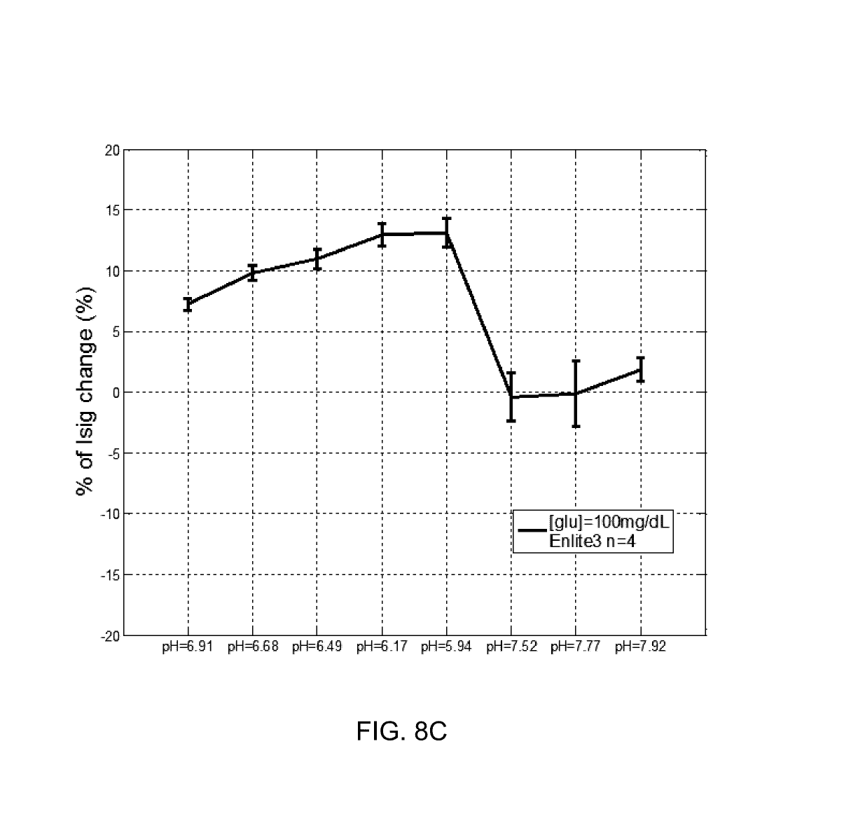

As noted above, in glucose enzymatic reactions, generated products such as Gluconic Acid and Hydrogen Peroxide could negatively impact enzyme activity thus compromise glucose concentration reading. In order to get an accurate glucose concentration reading, a calibration curve of amperometric current versus pH can be used to address the current shift caused by pH change. There are a number of ways in which algorithms can compensate for changes in pH. Optionally, for example, there are two calibration curves that the algorithm would use: OCP VS. pH (see, e.g. FIG. 8A) and Isig VS. pH change (see, e.g. FIG. 8B). These two curves can utilize values based on empirical sensor data that shows the relationship between current and pH. During sensor operation in vivo: pH sensor can continuously monitor the Open Circuit Potential. Once the OCP reaches a certain threshold such as lower than 20 mV (˜pH=8) and higher than 180 mV (˜pH=6), compensation algorithm can be switched on. It can find the corresponding pH based on OCP reading, and perform the Isig compensation based on the pH.

In some embodiments of the invention, the pH electrode and/or the working electrode comprises iridium oxide. Iridium Oxide (IrOx), has highly desirable characteristics such as biocompatibility, stability over a large range of pH, and fast response time to pH changes. Initial characterization of the IrOx based pH sensor demonstrated a linear response to pH (r2>False 95%) and a sensitivity of =50 mV/pH. In some embodiments of the invention, the pH electrode functions as the working electrode. In some embodiments of the invention, the working electrode comprises platinum black coated with a glucose oxidase composition that forms gluconic acid and hydrogen peroxide in the presence of glucose. In embodiments of the invention, the pH electrode and the working electrode can both be in operable contact with the reference electrode and the counter electrode. In certain embodiments, the pH electrode continuously monitors the open circuit potential between the pH electrode and the reference electrode.

Electroplating IrOx is a simple, low cost, short-preparation-time process. IrOx film can be plated on a variety of materials and platforms and it can be used in a wide range of applications. Electroplated IrOx film is sensitive to pH change and is catalytic to break down hydrogen peroxide (H₂O₂) as well. In a 3-electrode electrochemical cell, electroplated IrOx can function as a sensitive working electrode, and/or a stable reference electrode. On a redundant-electrode platform, multiple glucose sensing and or pH sensing electrodes can provide more accurate readings by averaged signals or additional information from different signals. In certain dual sensing embodiments, a micro-fabricated electrochemical cell with two electroplated IrOx working electrodes can be used. IrOx film has both sensitive pH sensing property and stable glucose sensing capability. Therefore one IrOx working electrode can be used for pH sensing where open circuit potential (OCP) is measured between working electrode and reference electrode, while the other independent working electrode can be used for glucose sensing where the H₂O₂ oxidation current is measured.

Certain embodiments of the invention use a single electrode dual mode sensing approach. In such embodiments, a micro-fabricated electrochemical cell with one electroplated IrOx working electrodes can be used, as IrOx film has both sensitive pH sensing property and stable glucose sensing capability. The IrOx working electrode can be periodically biased at a certain working potential to measure glucose concentration. At other times, the IrOx working electrode can be used for pH sensing (when Open Circuit Potential (OCP) is measured between working electrode and reference electrode).

Certain embodiments of the invention use electrodes formed from and/or coated with different layers of the materials disclosed herein. In one illustrative embodiment, two electrodes are disposed together on one substrate. One electrode is plated IrOx+GOx with no outer analyte modulating membrane while the other (also plated IrOx) has an analyte modulating membrane (e.g. a glucose limiting polymer) of well-defined thickness deposited over it. Optionally, the sensor includes an electrode formed from IrOx and an electrode formed from Platinum and different signals at these two electrodes are used for the detection of interfering molecules (e.g. acetaminophen). In yet another embodiment of the invention, a micro-fabricated electrochemical cell with an electroplated IrOx working electrode can be used. In this embodiment, potential differences between working electrode and reference electrode are used to assess the pH of the local environment.

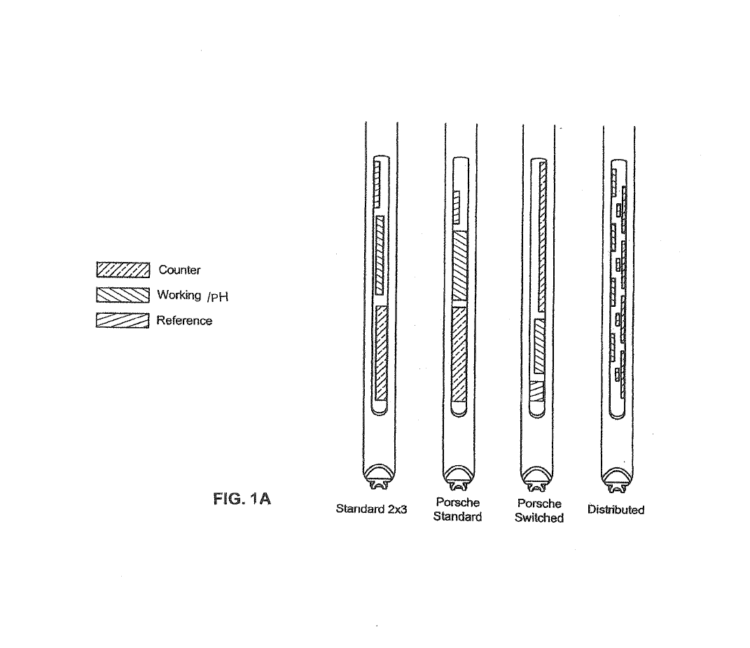

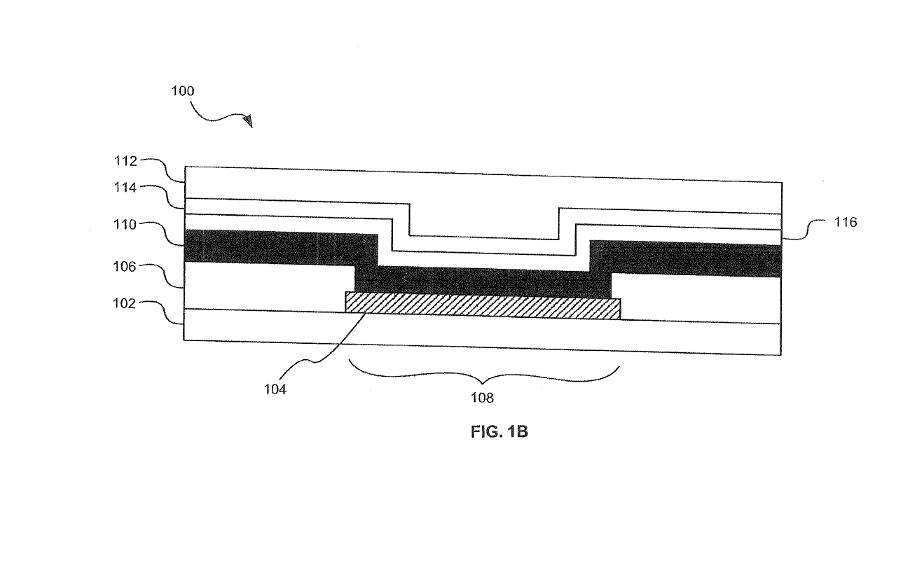

Typical sensors that include such adhesion promoting layers include amperometric glucose sensors that comprise glucose oxidase (e.g. within an analyte sensing layer) disposed over a one or more electrodes. A schematic of such embodiments is shown in FIG. 1B. In some embodiments of the invention, the layers are organized so that an analyte sensing layer is disposed over a conductive layer and the adhesion promoting layer disposed over the analyte sensing layer. In certain embodiments, the adhesion promoting layer is in direct contact with materials in the protein layer on a first side and in direct contact with materials in the analyte modulating layer on a second side. In other embodiments, the adhesion promoting layer is in direct contact with materials in the analyte sensing layer on a first side and in direct contact with materials in the analyte modulating layer on a second side. In some embodiments of the invention, the analyte sensing layer comprises an enzyme selected from the group consisting of glucose oxidase, glucose dehydrogenase, lactate oxidase, hexokinase and lactose dehydrogenase.

In some embodiments, the adhesion promoting layer is disposed over a protein layer that is disposed over the analyte sensing layer, for example a protein layer comprising bovine serum albumin (BSA) or human serum albumin (HSA). In typical embodiments, the protein constituent in this layer comprises an albumin such as human serum albumin. The HSA concentration may vary between about 0.5%-30% (w/v). Typically the HSA concentration is about 1-10% w/v, and most typically is about 5% w/v. In alternative embodiments of the invention, collagen or BSA or other structural proteins used in these contexts can be used instead of or in addition to HSA. Embodiments on the invention include further layers disposed over the adhesion promoting layer, for example an analyte modulating layer. In certain embodiments of the invention, an analyte modulating layer comprises an isocyanate compound and the isocyanate comprises an atom is covalently coupled to an atom in an allylamine in the adhesion promoting layer.

In certain embodiments of the invention, the analyte modulating layer comprises a linear polyurethane/polyurea polymer. Typically, the analyte modulating layer is formed from a mixture comprising: a diisocyanate compound (typically about 50 mol % of the reactants in the mixture); at least one hydrophilic diol or hydrophilic diamine compound (typically about 17 to 45 mol % of the reactants in the mixture); and a siloxane compound. Optionally the polyurethane/polyurea polymer comprises 45-55 mol % (e.g. 50 mol %) of a diisocyanate (e.g. 4,4′-diisocyanate), 10-20 (e.g. 12.5 mol %) mol % of a siloxane (e.g. polymethylhydrosiloxane, trimethylsilyl terminated), and 30-45 mol % (e.g. 37.5 mol %) of a hydrophilic diol or hydrophilic diamine compound (e.g. polypropylene glycol diamine having an average molecular weight of 600 Daltons, Jeffamine 600). In certain embodiments of the analyte modulating layer a first polyurethane/polyurea polymer is blended with a second polymer formed from a mixture comprising: 5-45 weight % of a 2-(dimethylamino)ethyl methacrylate compound; 15-55 weight % of a methyl methacrylate compound; 15-55 weight % of a polydimethyl siloxane monomethacryloxypropyl compound; 5-35 weight % of a poly(ethylene oxide) methyl ether methacrylate compound; and 1-20 weight % 2-hydroxyethyl methacrylate, with the first polymer and the second polymer blended together at a ratio between 1:1 and 1:20 weight %.

In some embodiments of the invention, the analyte modulating layer can comprise a blended mixture of a polyurethane/polyurea polymer formed from a mixture comprising a diisocyanate; a hydrophilic polymer comprising a hydrophilic diol or hydrophilic diamine; and a siloxane having an amino, hydroxyl or carboxylic acid functional group at a terminus. Optionally this polyurethane/polyurea polymer is blended with a branched acrylate polymer formed from a mixture comprising a butyl, propyl, ethyl or methyl-acrylate; an amino-acrylate; a siloxane-acrylate; and a poly(ethylene oxide)-acrylate. Optionally the analyte modulating layer exhibits a water adsorption profile of 40-60% of membrane weight. In certain embodiments of the invention, the analyte modulating layer is 5-15 um thick. In some embodiments, the analyte modulating layer comprises a polyurethane/polyurea polymer formed from a mixture comprising: a diisocyanate; a hydrophilic polymer comprising a hydrophilic diol or hydrophilic diamine; a siloxane having an amino, hydroxyl or carboxylic acid functional group at a terminus; and a polyurethane/polyurea polymer stabilizing compound selected for its ability to inhibit thermal and oxidative degradation of polyurethane/polyurea polymers formed from the mixture, wherein the polyurethane/polyurea polymer stabilizing compound has a molecular weight of less than 1000 g/mol; and comprises a benzyl ring having a hydroxyl moiety (ArOH). In typical embodiments of the invention, the polyurethane/polyurea polymer stabilizing compound exhibits an antioxidant activity (e.g. embodiments that comprise phenolic antioxidants). Optionally, the polyurethane/polyurea polymer stabilizing compound comprises at least two benzyl rings having a hydroxyl moiety.

### Illustrative Sensor Components and Systems of the Invention

In typical embodiments of the invention, electrochemical sensors are operatively coupled to a sensor input capable of receiving signals from the electrochemical sensor; and a processor coupled to the sensor input, wherein the processor is capable of characterizing one or more signals received from the electrochemical sensor. In certain embodiments of the invention, the electrical conduit of the electrode is coupled to a potentiostat. Optionally, a pulsed voltage is used to obtain a signal from an electrode. In certain embodiments of the invention, the processor is capable of comparing a first signal received from a working electrode in response to a first working potential with a second signal received from a working electrode in response to a second working potential. Optionally, the electrode is coupled to a processor adapted to convert data obtained from observing fluctuations in electrical current from a first format into a second format. Such embodiments include, for example, processors designed to convert a sensor current Input Signal (e.g. ISIG measured in nA) to a blood glucose concentration.

In many embodiments of the invention, the sensors comprise a biocompatible region adapted to be implanted in vivo. In some embodiments, the sensor comprises a discreet probe that pierces an in vivo environment. In embodiments of the invention, the biocompatible region can comprise a polymer that contacts an in vivo tissue. Optionally, the polymer is a hydrophilic polymer (e.g. one that absorbs water). In this way, sensors used in the systems of the invention can be used to sense a wide variety of analytes in different aqueous environments. In some embodiments of the invention, the electrode is coupled to a piercing member (e.g. a needle) adapted to be implanted in vivo. While sensor embodiments of the invention can comprise one or two piercing members, optionally such sensor apparatuses can include 3 or 4 or 5 or more piercing members that are coupled to and extend from a base element and are operatively coupled to 3 or 4 or 5 or more electrochemical sensors (e.g. microneedle arrays, embodiments of which are disclosed for example in U.S. Pat. Nos. 7,291,497 and 7,027,478, and U.S. patent Application No. 20080015494, the contents of which are incorporated by reference).

Embodiments of the invention include analyte sensor apparatus designed to utilize the compositions disclosed herein. Such apparatuses typically include a base on which an electrode is formed (e.g. an array of electrically conductive members configured to form a working electrode). Optionally this base comprises a plurality of indentations and the plurality of electrically conductive members are individually positioned within the plurality of indentations and the electrically conductive members comprise an electroactive surface adapted to sense fluctuations in electrical current at the electroactive surface.

In some embodiments of the invention where an electrode is formed from an array of electrically conductive members, the plurality of electrically conductive members are formed from shapes selected to avoid sharp edges and corners, electrode structures where electric charges can accumulate. In typical embodiments of the invention, the electrically conductive members can be formed to exhibit an ellipsoid geometry. For example, in some embodiments of the invention, the electrically conductive members comprise ellipses, circular discs, or combinations of ellipses and circular discs. Typically, such electrically conductive members are formed to have a diameter of at least 1 μm, for example, a diameter from 1 μm to 100 μm (e.g. circular discs having a diameter of 30, 40 or 50 μm). Optionally, the array comprises at least 5, 10, 20, 50 or 100 electrically conductive members.

In some embodiments of the invention, the array of electrically conductive members is coupled to a common electrical conduit (e.g. so that the conductive members of the array are not separately wired, and are instead electrically linked as a group). Optionally, the electrical conduit is coupled to a power source adapted to sense fluctuations in electrical current of the array of the working electrode. Typically the apparatus include a reference electrode; and a counter electrode. Optionally one or more of these electrodes also comprises a plurality of electrically conductive members disposed on the base in an array. In some embodiments, each of the electrically conductive members of the electrode (e.g. the counter electrode) comprises an electroactive surface adapted to sense fluctuations in electrical current at the electroactive surface; and the group of electrically conductive members are coupled to a power source (e.g. a potentiostat or the like).

In some embodiments of the invention, the apparatus comprises a plurality of working electrodes, counter electrodes and reference electrodes clustered together in units consisting essentially of one working electrode, one counter electrode and one reference electrode; and the clustered units are longitudinally distributed on the base layer in a repeating pattern of units. In some sensor embodiments, the distributed electrodes are organized/disposed within a flex-circuit assembly (i.e. a circuitry assembly that utilizes flexible rather than rigid materials). Such flex-circuit assembly embodiments provide an interconnected assembly of elements (e.g. electrodes, electrical conduits, contact pads and the like) configured to facilitate wearer comfort (for example by reducing pad stiffness and wearer discomfort).

Typically, the sensor electrodes of the invention are coated with a plurality of materials having properties that, for example, facilitate analyte sensing. In some embodiments of the invention, an analyte sensing layer is disposed over electrically conductive members, and includes an agent that is selected for its ability to detectably alter the electrical current at the working electrode in the presence of an analyte. In the working embodiments of the invention that are disclosed herein, the agent is glucose oxidase, a protein that undergoes a chemical reaction in the presence of glucose that results in an alteration in the electrical current at the working electrode. These working embodiments further include an analyte modulating layer disposed over the analyte sensing layer, wherein the analyte modulating layer modulates the diffusion of glucose as it migrates from an in vivo environment to the analyte sensing layer. In certain embodiments of the invention, the analyte modulating layer comprises a hydrophilic comb-copolymer having a central chain and a plurality of side chains coupled to the central chain, wherein at least one side chain comprises a silicone moiety. In certain embodiments of the invention, the analyte modulating layer comprises a blended mixture of: a linear polyurethane/polyurea polymer, and a branched acrylate polymer; and the linear polyurethane/polyurea polymer and the branched acrylate polymer are blended at a ratio of between 1:1 and 1:20 (e.g. 1:2) by weight %. Typically, this analyte modulating layer composition comprises a first polymer formed from a mixture comprising a diisocyanate; at least one hydrophilic diol or hydrophilic diamine; and a siloxane; that is blended with a second polymer formed from a mixture comprising: a 2-(dimethylamino) ethyl methacrylate; a methyl methacrylate; a polydimethyl siloxane monomethacryloxypropyl; a poly(ethylene oxide) methyl ether methacrylate; and a 2-hydroxyethyl methacrylate. Additional material layers can be included in such apparatuses. For example, in some embodiments of the invention, the apparatus comprises an adhesion promoting layer disposed between the analyte sensing layer and the analyte modulating layer.

Embodiments of the invention include dry plasma processes form making adhesion promoting (AP) layers in sensors comprising a plurality of layered materials (see, e.g. International Patent Application No. PCT/US2013/049138). The dry plasma processes disclosed PCT/US2013/049138 have a number of advantages over conventional wet chemistry processes used to form adhesion promoting layers, including reducing and/or eliminating the use of certain hazardous compounds, thereby reducing toxic wastes that can result from such processes. Embodiments of the invention also include adhesion promoting compositions formed from these processes, compositions that exhibit a combination of desirable material properties including relatively thin and highly uniform structural profiles.

One sensor embodiment shown in FIG. 1 is a amperometric sensor 100 having a plurality of layered elements including a base layer 102, a conductive layer 104 (e.g. one comprising the plurality of electrically conductive members) which is disposed on and/or combined with the base layer 102. Typically the conductive layer 104 comprises one or more electrodes. An analyte sensing layer 110 (typically comprising an enzyme such as glucose oxidase) is disposed on one or more of the exposed electrodes of the conductive layer 104. A protein layer 116 disposed upon the analyte sensing layer 110. An analyte modulating layer 112 is disposed above the analyte sensing layer 110 to regulate analyte (e.g. glucose) access with the analyte sensing layer 110. An adhesion promoter layer 114 is disposed between layers such as the analyte modulating layer 112 and the analyte sensing layer 110 as shown in FIG. 1 in order to facilitate their contact and/or adhesion. This embodiment also comprises a cover layer 106 such as a polymer coating can be disposed on portions of the sensor 100. Apertures 108 can be formed in one or more layers of such sensors. Amperometric glucose sensors having this type of design are disclosed, for example, in U.S. Patent Application Publication Nos. 20070227907, 20100025238, 20110319734 and 20110152654, the contents of each of which are incorporated herein by reference.

Yet another embodiment of the invention is a method of sensing an analyte within the body of a mammal. Typically this method comprises implanting an analyte sensor having a pH sensor electrode disclosed herein within the mammal (e.g. in the interstitial space of a diabetic individual), sensing an alteration in current at the working electrode in the presence of the analyte at different pH levels; and then correlating the alteration in current with the presence of the analyte, so that the analyte is sensed.

Embodiments of the invention also provide articles of manufacture and kits for observing a concentration of an analyte. In an illustrative embodiment, the kit includes a sensor comprising a composition as disclosed herein. In typical embodiments, the sensors are disposed in the kit within a sealed sterile dry package. Optionally the kit comprises an insertion device that facilitates insertion of the sensor. The kit and/or sensor set typically comprises a container, a label and an analyte sensor as described above. Suitable containers include, for example, an easy to open package made from a material such as a metal foil, bottles, vials, syringes, and test tubes. The containers may be formed from a variety of materials such as metals (e.g. foils) paper products, glass or plastic. The label on, or associated with, the container indicates that the sensor is used for assaying the analyte of choice. The kit and/or sensor set may include other materials desirable from a commercial and user standpoint, including buffers, diluents, filters, needles, syringes, and package inserts with instructions for use.

Specific aspects of embodiments of the invention are discussed in detail in the following sections.

### Typical Elements, Configurations and Analyte Sensor Embodiments of the Invention

### A. Typical Elements Found in of Embodiments of the Invention

FIG. 1 illustrates a cross-section of a typical sensor embodiment 100 of the present invention. This sensor embodiment is formed from a plurality of components that are typically in the form of layers of various conductive and non-conductive constituents disposed on each other according to art accepted methods and/or the specific methods of the invention disclosed herein. The components of the sensor are typically characterized herein as layers because, for example, it allows for a facile characterization of the sensor structure shown in FIG. 1. Artisans will understand however, that in certain embodiments of the invention, the sensor constituents are combined such that multiple constituents form one or more heterogeneous layers. In this context, those of skill in the art understand that the ordering of the layered constituents can be altered in various embodiments of the invention.

The embodiment shown in FIG. 1 includes a base layer 102 to support the sensor 100. The base layer 102 can be made of a material such as a metal and/or a ceramic and/or a polymeric substrate, which may be self-supporting or further supported by another material as is known in the art. Embodiments of the invention include a conductive layer 104 which is disposed on and/or combined with the base layer 102. Typically the conductive layer 104 comprises one or more electrically conductive elements that function as electrodes. An operating sensor 100 typically includes a plurality of electrodes such as a working electrode, a counter electrode and a reference electrode. Other embodiments may also include a plurality of working and/or counter and/or reference electrodes and/or one or more electrodes that performs multiple functions, for example one that functions as both as a reference and a counter electrode.

As discussed in detail below, the base layer 102 and/or conductive layer 104 can be generated using many known techniques and materials. In certain embodiments of the invention, the electrical circuit of the sensor is defined by etching the disposed conductive layer 104 into a desired pattern of conductive paths. A typical electrical circuit for the sensor 100 comprises two or more adjacent conductive paths with regions at a proximal end to form contact pads and regions at a distal end to form sensor electrodes. An electrically insulating cover layer 106 such as a polymer coating can be disposed on portions of the sensor 100. Acceptable polymer coatings for use as the insulating protective cover layer 106 can include, but are not limited to, non-toxic biocompatible polymers such as silicone compounds, polyimides, biocompatible solder masks, epoxy acrylate copolymers, or the like. In the sensors of the present invention, one or more exposed regions or apertures 108 can be made through the cover layer 106 to open the conductive layer 104 to the external environment and to, for example, allow an analyte such as glucose to permeate the layers of the sensor and be sensed by the sensing elements. Apertures 108 can be formed by a number of techniques, including laser ablation, tape masking, chemical milling or etching or photolithographic development or the like. In certain embodiments of the invention, during manufacture, a secondary photoresist can also be applied to the protective layer 106 to define the regions of the protective layer to be removed to form the aperture(s) 108. The exposed electrodes and/or contact pads can also undergo secondary processing (e.g. through the apertures 108), such as additional plating processing, to prepare the surfaces and/or strengthen the conductive regions.

In the sensor configuration shown in FIG. 1, an analyte sensing layer 110 is disposed on one or more of the exposed electrodes of the conductive layer 104. Typically, the analyte sensing layer 110 comprises an enzyme capable of producing and/or utilizing oxygen and/or hydrogen peroxide (for example glucose oxidase). Optionally the enzyme in the analyte sensing layer is combined with a carrier protein such as human serum albumin, bovine serum albumin or the like. In an illustrative embodiment, an oxidoreductase enzyme such as glucose oxidase in the analyte sensing layer 110 reacts with glucose to produce hydrogen peroxide, a compound which then modulates a current at an electrode. As this modulation of current depends on the concentration of hydrogen peroxide, and the concentration of hydrogen peroxide correlates to the concentration of glucose, the concentration of glucose can be determined by monitoring this modulation in the current. In a specific embodiment of the invention, the hydrogen peroxide is oxidized at a working electrode which is an anode (also termed herein the anodic working electrode), with the resulting current being proportional to the hydrogen peroxide concentration. Such modulations in the current caused by changing hydrogen peroxide concentrations can by monitored by any one of a variety of sensor detector apparatuses such as a universal sensor amperometric biosensor detector or one of the other variety of similar devices known in the art such as glucose monitoring devices produced by Medtronic Diabetes.

In embodiments of the invention, the analyte sensing layer 110 can be applied over portions of the conductive layer or over the entire region of the conductive layer. Typically the analyte sensing layer 110 is disposed on the working electrode which can be the anode or the cathode. Optionally, the analyte sensing layer 110 is also disposed on a counter and/or reference electrode. Methods for generating a thin analyte sensing layer 110 include brushing the layer onto a substrate (e.g. the reactive surface of a platinum black electrode), as well as spin coating processes, dip and dry processes, low shear spraying processes, ink-jet printing processes, silk screen processes and the like. In certain embodiments of the invention, brushing is used to: (1) allow for a precise localization of the layer; and (2) push the layer deep into the architecture of the reactive surface of an electrode (e.g. platinum black produced by an electrodeposition process).

Typically, the analyte sensing layer 110 is coated and or disposed next to one or more additional layers. Optionally, the one or more additional layers includes a protein layer 116 disposed upon the analyte sensing layer 110. Typically, the protein layer 116 comprises a protein such as human serum albumin, bovine serum albumin or the like. Typically, the protein layer 116 comprises human serum albumin. In some embodiments of the invention, an additional layer includes an analyte modulating layer 112 that is disposed above the analyte sensing layer 110 to regulate analyte contact with the analyte sensing layer 110. For example, the analyte modulating membrane layer 112 can comprise a glucose limiting membrane, which regulates the amount of glucose that contacts an enzyme such as glucose oxidase that is present in the analyte sensing layer. Such glucose limiting membranes can be made from a wide variety of materials known to be suitable for such purposes, e.g., silicone compounds such as polydimethyl siloxanes, polyurethanes, polyurea cellulose acetates, Nafion, polyester sulfonic acids (e.g. Kodak AQ), hydrogels or any other suitable hydrophilic membranes known to those skilled in the art.

In typical embodiments of the invention, an adhesion promoter layer 114 is disposed between the analyte modulating layer 112 and the analyte sensing layer 110 as shown in FIG. 1 in order to facilitate their contact and/or adhesion. In a specific embodiment of the invention, an adhesion promoter layer 114 is disposed between the analyte modulating layer 112 and the protein layer 116 as shown in FIG. 1 in order to facilitate their contact and/or adhesion. The adhesion promoter layer 114 can be made from any one of a wide variety of materials known in the art to facilitate the bonding between such layers. Typically, the adhesion promoter layer 114 comprises a silane compound. In alternative embodiments, protein or like molecules in the analyte sensing layer 110 can be sufficiently crosslinked or otherwise prepared to allow the analyte modulating membrane layer 112 to be disposed in direct contact with the analyte sensing layer 110 in the absence of an adhesion promoter layer 114.

### B. Typical Analyte Sensor Constituents Used in Embodiments of the Invention

The following disclosure provides examples of typical elements/constituents used in sensor embodiments of the invention. While these elements can be described as discreet units (e.g. layers), those of skill in the art understand that sensors can be designed to contain elements having a combination of some or all of the material properties and/or functions of the elements/constituents discussed below (e.g. an element that serves both as a supporting base constituent and/or a conductive constituent and/or a matrix for the analyte sensing constituent and which further functions as an electrode in the sensor). Those in the art understand that these thin film analyte sensors can be adapted for use in a number of sensor systems such as those described below.

### Base Constituent

Sensors of the invention typically include a base constituent (see, e.g. element 102 in FIG. 1). The term “base constituent” is used herein according to art accepted terminology and refers to the constituent in the apparatus that typically provides a supporting matrix for the plurality of constituents that are stacked on top of one another and comprise the functioning sensor. In one form, the base constituent comprises a thin film sheet of insulative (e.g. electrically insulative and/or water impermeable) material. This base constituent can be made of a wide variety of materials having desirable qualities such as dielectric properties, water impermeability and hermeticity. Some materials include metallic, and/or ceramic and/or polymeric substrates or the like.

### Conductive Constituent

The electrochemical sensors of the invention typically include a conductive constituent disposed upon the base constituent that includes at least one electrode for contacting an analyte or its byproduct (e.g. oxygen and/or hydrogen peroxide) to be assayed (see, e.g. element 104 in FIG. 1). The term “conductive constituent” is used herein according to art accepted terminology and refers to electrically conductive sensor elements such as a plurality of electrically conductive members disposed on the base layer (e.g. so as to form a microarray electrode) and which are capable of measuring a detectable signal and conducting this to a detection apparatus. An illustrative example of this is a conductive constituent that forms a working electrode that can measure an increase or decrease in current in response to exposure to a stimuli such as the change in the concentration of an analyte or its byproduct as compared to a reference electrode that does not experience the change in the concentration of the analyte, a coreactant (e.g. oxygen) used when the analyte interacts with a composition (e.g. the enzyme glucose oxidase) present in analyte sensing constituent 110 or a reaction product of this interaction (e.g. hydrogen peroxide). Illustrative examples of such elements include electrodes which are capable of producing variable detectable signals in the presence of variable concentrations of molecules such as hydrogen peroxide or oxygen.

In addition to the working electrode and the working electrode, the analyte sensors of the invention typically include a reference electrode or a combined reference and counter electrode (also termed a quasi-reference electrode or a counter/reference electrode). If the sensor does not have a counter/reference electrode then it may include a separate counter electrode, which may be made from the same or different materials as the working electrode and/or the pH electrode. Typical sensors of the present invention have one or more working and/or pH electrodes and one or more counter, reference, and/or counter/reference electrodes. One embodiment of the sensor of the present invention has two, three or four or more working electrodes. These working electrodes in the sensor may be integrally connected or they may be kept separate. Optionally, the electrodes can be disposed on a single surface or side of the sensor structure. Alternatively, the electrodes can be disposed on a multiple surfaces or sides of the sensor structure (and can for example be connected by vias through the sensor material(s) to the surfaces on which the electrodes are disposed). In certain embodiments of the invention, the reactive surfaces of the electrodes are of different relative areas/sizes, for example a 1× reference electrode, a 2.6× working electrode and a 3.6× counter electrode.

### Interference Rejection Constituent

The electrochemical sensors of the invention optionally include an interference rejection constituent disposed between the surface of the electrode and the environment to be assayed. In particular, certain sensor embodiments rely on the oxidation and/or reduction of hydrogen peroxide generated by enzymatic reactions on the surface of a working electrode at a constant potential applied. Because amperometric detection based on direct oxidation of hydrogen peroxide requires a relatively high oxidation potential, sensors employing this detection scheme may suffer interference from oxidizable species that are present in biological fluids such as ascorbic acid, uric acid and acetaminophen. In this context, the term “interference rejection constituent” is used herein according to art accepted terminology and refers to a coating or membrane in the sensor that functions to inhibit spurious signals generated by such oxidizable species which interfere with the detection of the signal generated by the analyte to be sensed. Certain interference rejection constituents function via size exclusion (e.g. by excluding interfering species of a specific size). Examples of interference rejection constituents include one or more layers or coatings of compounds such as hydrophilic polyurethanes, cellulose acetate (including cellulose acetate incorporating agents such as poly(ethylene glycol), polyethersulfones, polytetra-fluoroethylenes, the perfluoronated ionomer Nafion™, polyphenylenediamine, epoxy and the like.

### Analyte Sensing Constituent

The electrochemical sensors of the invention include an analyte sensing constituent disposed on the electrodes of the sensor (see, e.g. element 110 in FIG. 1). In working embodiments of the invention disclosed herein, this constituent comprises glucose oxidase. The term “analyte sensing constituent” is used herein according to art accepted terminology and refers to a constituent comprising a material that is capable of recognizing or reacting with an analyte whose presence is to be detected by the analyte sensor apparatus. Typically this material in the analyte sensing constituent produces a detectable signal after interacting with the analyte to be sensed, typically via the electrodes of the conductive constituent. In this regard the analyte sensing constituent and the electrodes of the conductive constituent work in combination to produce the electrical signal that is read by an apparatus associated with the analyte sensor. Typically, the analyte sensing constituent comprises an oxidoreductase enzyme capable of reacting with and/or producing a molecule whose change in concentration can be measured by measuring the change in the current at an electrode of the conductive constituent (e.g. oxygen and/or hydrogen peroxide), for example the enzyme glucose oxidase. An enzyme capable of producing a molecule such as hydrogen peroxide can be disposed on the electrodes according to a number of processes known in the art. The analyte sensing constituent can coat all or a portion of the various electrodes of the sensor. In this context, the analyte sensing constituent may coat the electrodes to an equivalent degree. Alternatively the analyte sensing constituent may coat different electrodes to different degrees, with for example the coated surface of the working electrode being larger than the coated surface of the counter and/or reference electrode.

Typical sensor embodiments of this element of the invention utilize an enzyme (e.g. glucose oxidase) that has been combined with a second protein (e.g. albumin) in a fixed ratio (e.g. one that is typically optimized for glucose oxidase stabilizing properties) and then applied on the surface of an electrode to form a thin enzyme constituent. In a typical embodiment, the analyte sensing constituent comprises a GOx and HSA mixture. In a typical embodiment of an analyte sensing constituent having GOx, the GOx reacts with glucose present in the sensing environment (e.g. the body of a mammal) and generates hydrogen peroxide.

As noted above, the enzyme and the second protein (e.g. an albumin) are typically treated to form a crosslinked matrix (e.g. by adding a cross-linking agent to the protein mixture). As is known in the art, crosslinking conditions may be manipulated to modulate factors such as the retained biological activity of the enzyme, its mechanical and/or operational stability. Illustrative crosslinking procedures are described in U.S. patent application Ser. No. 10/335,506 and PCT publication WO 03/035891 which are incorporated herein by reference. For example, an amine cross-linking reagent, such as, but not limited to, glutaraldehyde, can be added to the protein mixture. The addition of a cross-linking reagent to the protein mixture creates a protein paste. The concentration of the cross-linking reagent to be added may vary according to the concentration of the protein mixture. While glutaraldehyde is an illustrative crosslinking reagent, other cross-linking reagents may also be used or may be used in place of glutaraldehyde. Other suitable cross-linkers also may be used, as will be evident to those skilled in the art.

As noted above, in some embodiments of the invention, the analyte sensing constituent includes an agent (e.g. glucose oxidase) capable of producing a signal (e.g. a change in oxygen and/or hydrogen peroxide concentrations) that can be sensed by the electrically conductive elements (e.g. electrodes which sense changes in oxygen and/or hydrogen peroxide concentrations). However, other useful analyte sensing constituents can be formed from any composition that is capable of producing a detectable signal that can be sensed by the electrically conductive elements after interacting with a target analyte whose presence is to be detected. In some embodiments, the composition comprises an enzyme that modulates hydrogen peroxide concentrations upon reaction with an analyte to be sensed. Alternatively, the composition comprises an enzyme that modulates oxygen concentrations upon reaction with an analyte to be sensed. In this context, a wide variety of enzymes that either use or produce hydrogen peroxide and/or oxygen in a reaction with a physiological analyte are known in the art and these enzymes can be readily incorporated into the analyte sensing constituent composition. A variety of other enzymes known in the art can produce and/or utilize compounds whose modulation can be detected by electrically conductive elements such as the electrodes that are incorporated into the sensor designs described herein. Such enzymes include for example, enzymes specifically described in Table 1, pages 15-29 and/or Table 18, pages 111-112 of Protein Immobilization: Fundamentals and Applications (Bioprocess Technology, Vol 14) by Richard F. Taylor (Editor) Publisher: Marcel Dekker; Jan. 7, 1991) the entire contents of which are incorporated herein by reference.

### Protein Constituent

The electrochemical sensors of the invention optionally include a protein constituent disposed between the analyte sensing constituent and the analyte modulating constituent (see, e.g. element 116 in FIG. 1). The term “protein constituent” is used herein according to art accepted terminology and refers to constituent containing a carrier protein or the like that is selected for compatibility with the analyte sensing constituent and/or the analyte modulating constituent. In typical embodiments, the protein constituent comprises an albumin such as human serum albumin. The HSA concentration may vary between about 0.5%-30% (w/v). Typically the HSA concentration is about 1-10% w/v, and most typically is about 5% w/v. In alternative embodiments of the invention, collagen or BSA or other structural proteins used in these contexts can be used instead of or in addition to HSA. This constituent is typically crosslinked on the analyte sensing constituent according to art accepted protocols.

### Adhesion Promoting Constituent

The electrochemical sensors of the invention can include one or more adhesion promoting (AP) constituents (see, e.g. element 114 in FIG. 1). The term “adhesion promoting constituent” is used herein according to art accepted terminology and refers to a constituent that includes materials selected for their ability to promote adhesion between adjoining constituents in the sensor. Typically, the adhesion promoting constituent is disposed between the analyte sensing constituent and the analyte modulating constituent. Typically, the adhesion promoting constituent is disposed between the optional protein constituent and the analyte modulating constituent. The adhesion promoter constituent can be made from any one of a wide variety of materials known in the art to facilitate the bonding between such constituents and can be applied by any one of a wide variety of methods known in the art. Typically, the adhesion promoter constituent comprises a silane compound such as γ-aminopropyltrimethoxysilane.

### Analyte Modulating Constituent

The electrochemical sensors of the invention include an analyte modulating constituent disposed on the sensor (see, e.g. element 112 in FIG. 1). The term “analyte modulating constituent” is used herein according to art accepted terminology and refers to a constituent that typically forms a membrane on the sensor that operates to modulate the diffusion of one or more analytes, such as glucose, through the constituent. In certain embodiments of the invention, the analyte modulating constituent is an analyte-limiting membrane which operates to prevent or restrict the diffusion of one or more analytes, such as glucose, through the constituents. In other embodiments of the invention, the analyte-modulating constituent operates to facilitate the diffusion of one or more analytes, through the constituents. Optionally such analyte modulating constituents can be formed to prevent or restrict the diffusion of one type of molecule through the constituent (e.g. glucose), while at the same time allowing or even facilitating the diffusion of other types of molecules through the constituent (e.g. O₂).

With respect to glucose sensors, in known enzyme electrodes, glucose and oxygen from blood, as well as some interferants, such as ascorbic acid and uric acid, diffuse through a primary membrane of the sensor. As the glucose, oxygen and interferants reach the analyte sensing constituent, an enzyme, such as glucose oxidase, catalyzes the conversion of glucose to hydrogen peroxide and gluconolactone. The hydrogen peroxide may diffuse back through the analyte modulating constituent, or it may diffuse to an electrode where it can be reacted to form oxygen and a proton to produce a current that is proportional to the glucose concentration. The analyte modulating sensor membrane assembly serves several functions, including selectively allowing the passage of glucose therethrough (see, e.g. U.S. Patent Application No. 2011-0152654).

### Cover Constituent

The electrochemical sensors of the invention can include one or more cover constituents, which are typically electrically insulating protective constituents (see, e.g. element 106 in FIG. 1). Typically, such cover constituents can be in the form of a coating, sheath or tube and are disposed on at least a portion of the analyte modulating constituent. Acceptable polymer coatings for use as the insulating protective cover constituent can include, but are not limited to, non-toxic biocompatible polymers such as silicone compounds, polyimides, biocompatible solder masks, epoxy acrylate copolymers, or the like. Further, these coatings can be photo-imagable to facilitate photolithographic forming of apertures through to the conductive constituent. A typical cover constituent comprises spun on silicone. As is known in the art, this constituent can be a commercially available RTV (room temperature vulcanized) silicone composition. A typical chemistry in this context is polydimethyl siloxane (acetoxy based).

### C. Typical Analyte Sensor System Embodiments of the Invention

Embodiments of the sensor elements and sensors can be operatively coupled to a variety of other system elements typically used with analyte sensors (e.g. structural elements such as piercing members, insertion sets and the like as well as electronic components such as processors, monitors, medication infusion pumps and the like), for example to adapt them for use in various contexts (e.g. implantation within a mammal). One embodiment of the invention includes a method of monitoring a physiological characteristic of a user using an embodiment of the invention that includes an input element capable of receiving a signal from a sensor that is based on a sensed physiological characteristic value of the user, and a processor for analyzing the received signal. In typical embodiments of the invention, the processor determines a dynamic behavior of the physiological characteristic value and provides an observable indicator based upon the dynamic behavior of the physiological characteristic value so determined. In some embodiments, the physiological characteristic value is a measure of the concentration of blood glucose in the user. In other embodiments, the process of analyzing the received signal and determining a dynamic behavior includes repeatedly measuring the physiological characteristic value to obtain a series of physiological characteristic values in order to, for example, incorporate comparative redundancies into a sensor apparatus in a manner designed to provide confirmatory information on sensor function, analyte concentration measurements, the presence of interferences and the like.

Embodiments of the invention include devices which process display data from measurements of a sensed physiological characteristic (e.g. blood glucose concentrations) in a manner and format tailored to allow a user of the device to easily monitor and, if necessary, modulate the physiological status of that characteristic (e.g. modulation of blood glucose concentrations via insulin administration). An illustrative embodiment of the invention is a device comprising a sensor input capable of receiving a signal from a sensor, the signal being based on a sensed physiological characteristic value of a user; a memory for storing a plurality of measurements of the sensed physiological characteristic value of the user from the received signal from the sensor; and a display for presenting a text and/or graphical representation of the plurality of measurements of the sensed physiological characteristic value (e.g. text, a line graph or the like, a bar graph or the like, a grid pattern or the like or a combination thereof). Typically, the graphical representation displays real time measurements of the sensed physiological characteristic value. Such devices can be used in a variety of contexts, for example in combination with other medical apparatuses. In some embodiments of the invention, the device is used in combination with at least one other medical device (e.g. a glucose sensor).

An illustrative system embodiment consists of a glucose sensor, a transmitter and pump receiver and a glucose meter. In this system, radio signals from the transmitter can be sent to the pump receiver every 5 minutes to provide providing real-time sensor glucose (SG) values. Values/graphs are displayed on a monitor of the pump receiver so that a user can self monitor blood glucose and deliver insulin using their own insulin pump. Typically an embodiment of device disclosed herein communicates with a second medical device via a wired or wireless connection. Wireless communication can include for example the reception of emitted radiation signals as occurs with the transmission of signals via RF telemetry, infrared transmissions, optical transmission, sonic and ultrasonic transmissions and the like. Optionally, the device is an integral part of a medication infusion pump (e.g. an insulin pump). Typically in such devices, the physiological characteristic values include a plurality of measurements of blood glucose.

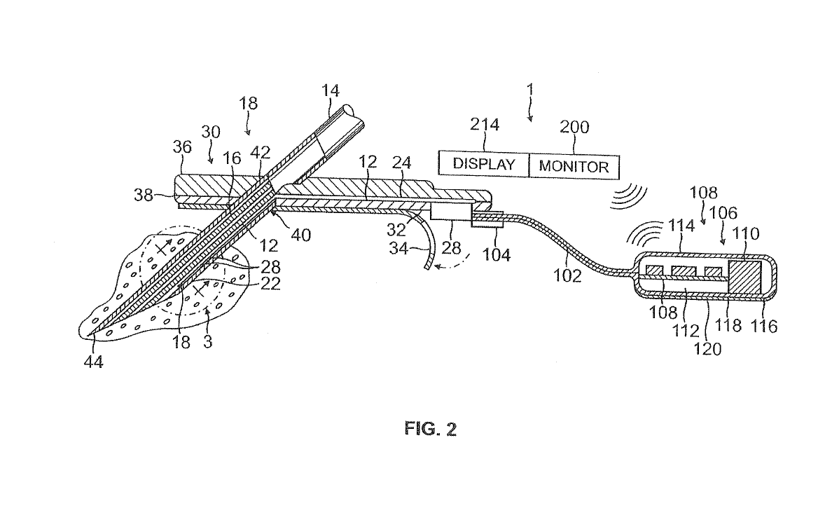

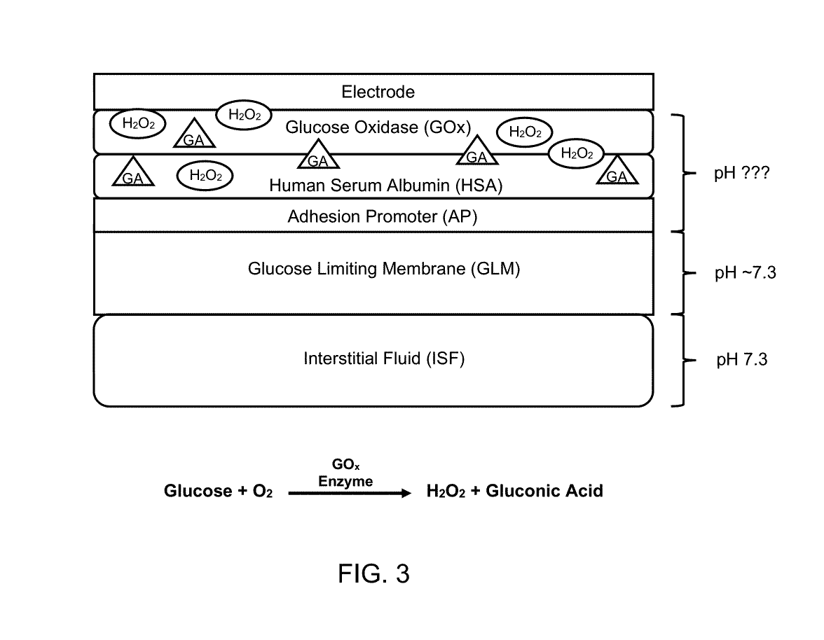

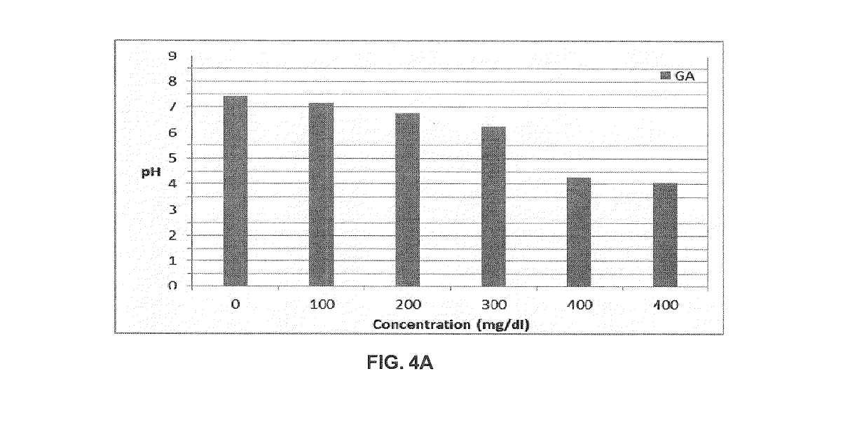

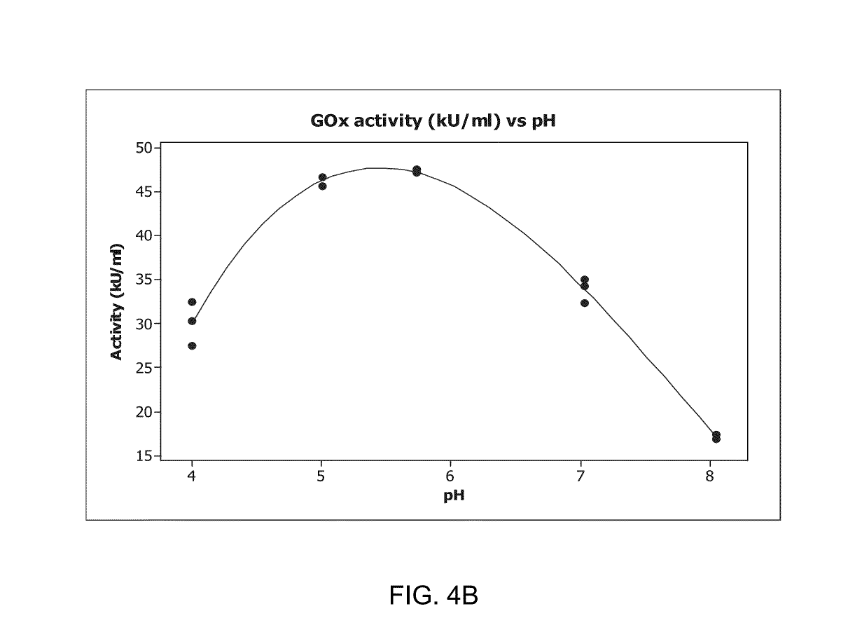

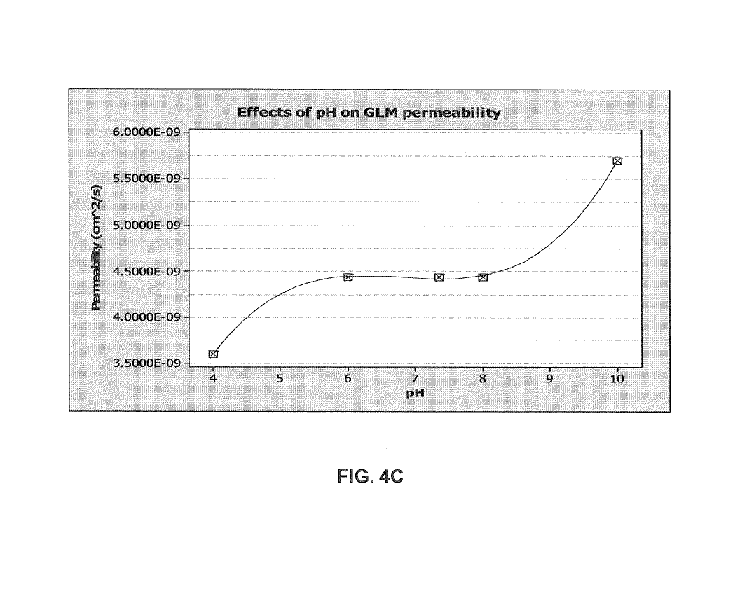

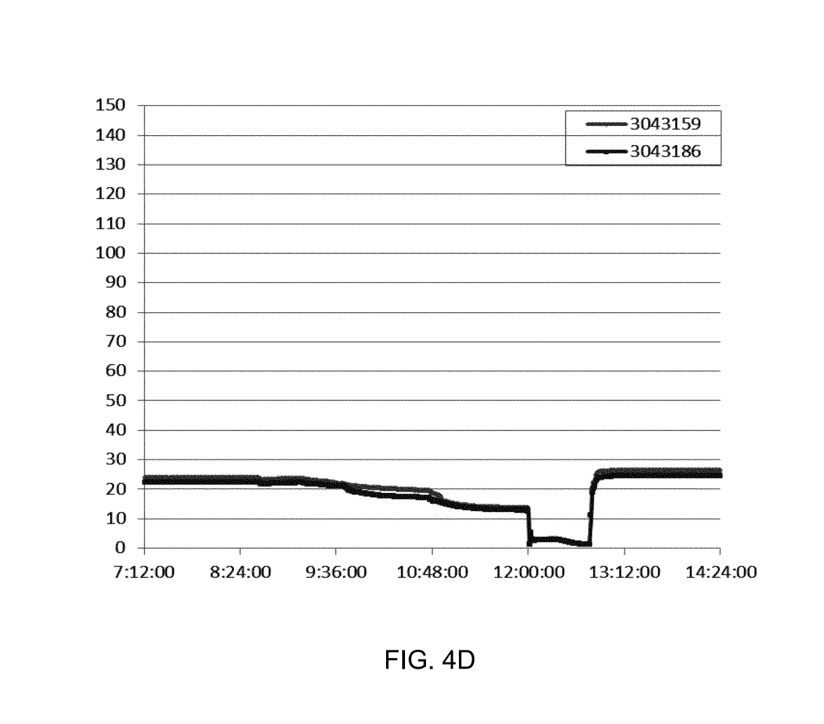

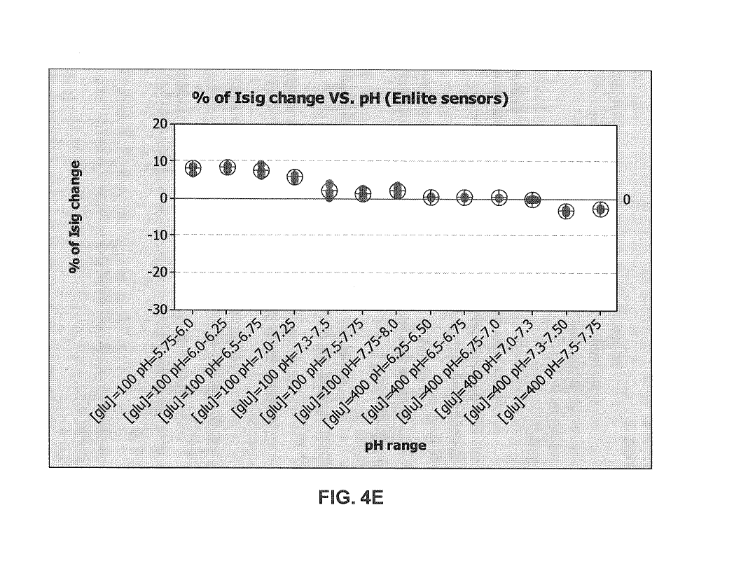

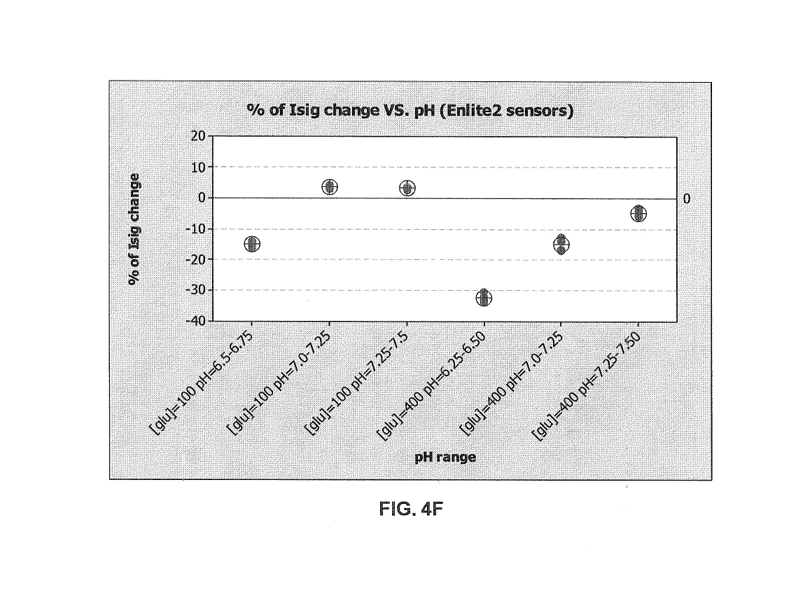

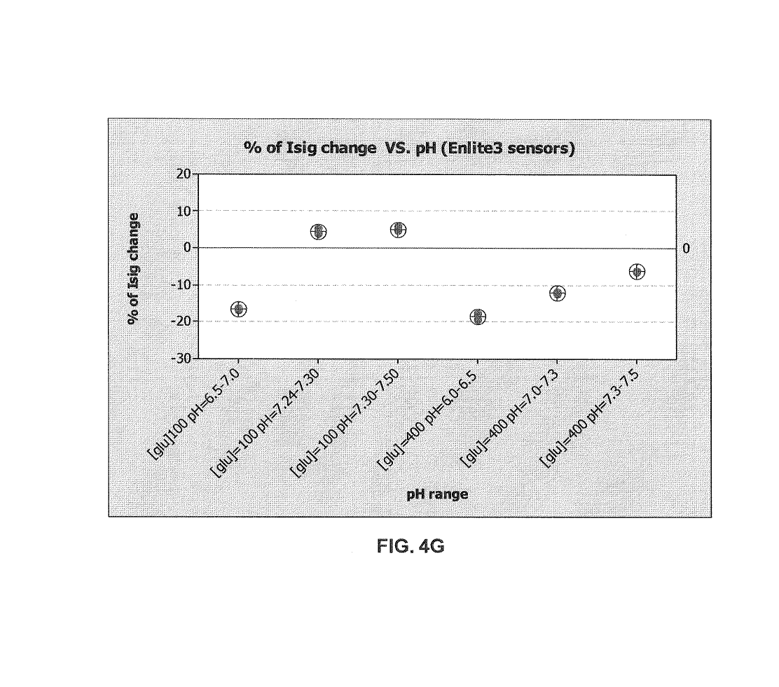

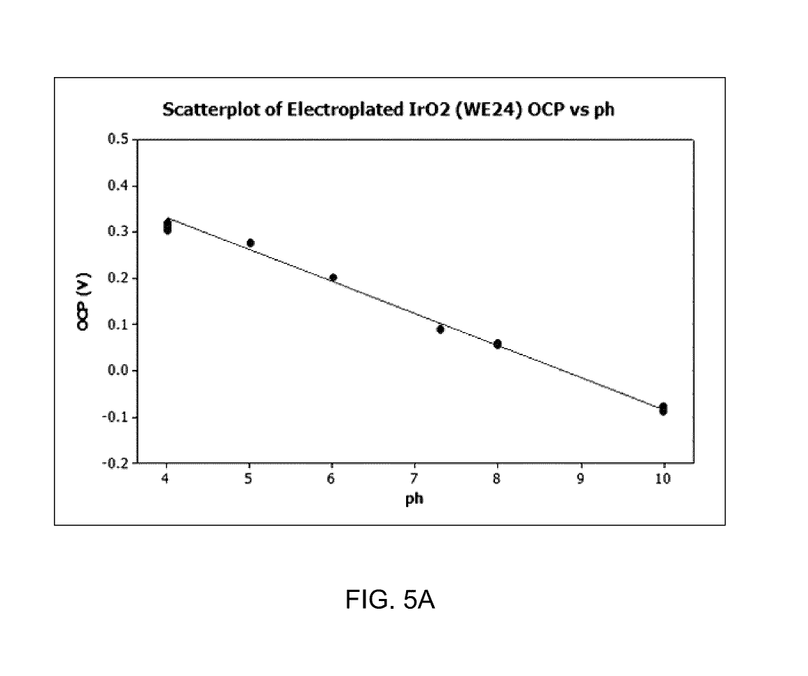

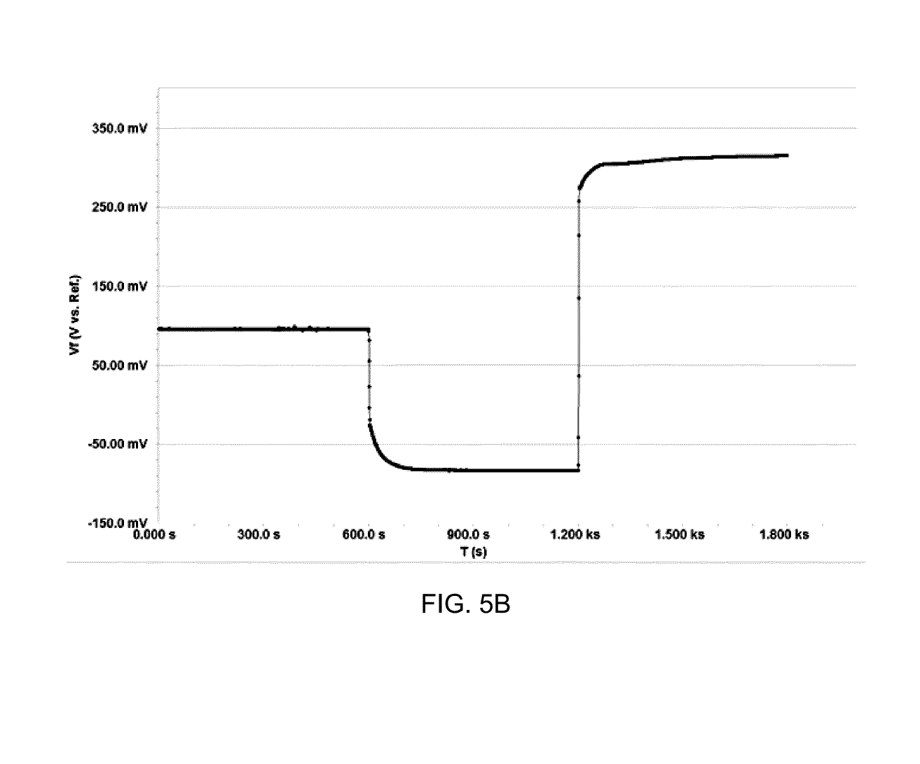

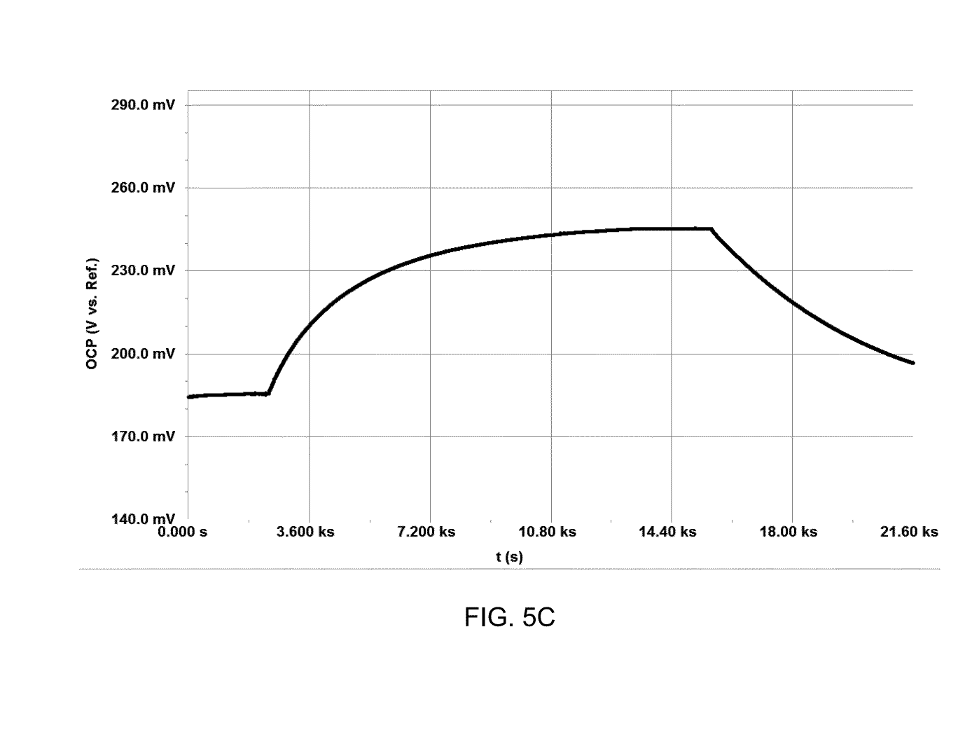

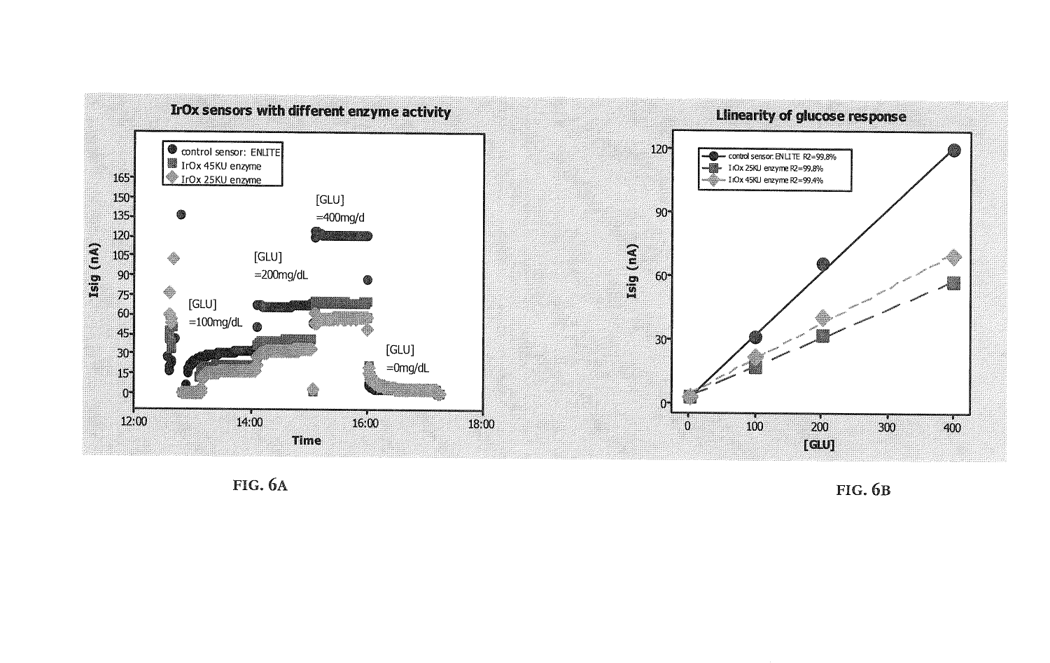

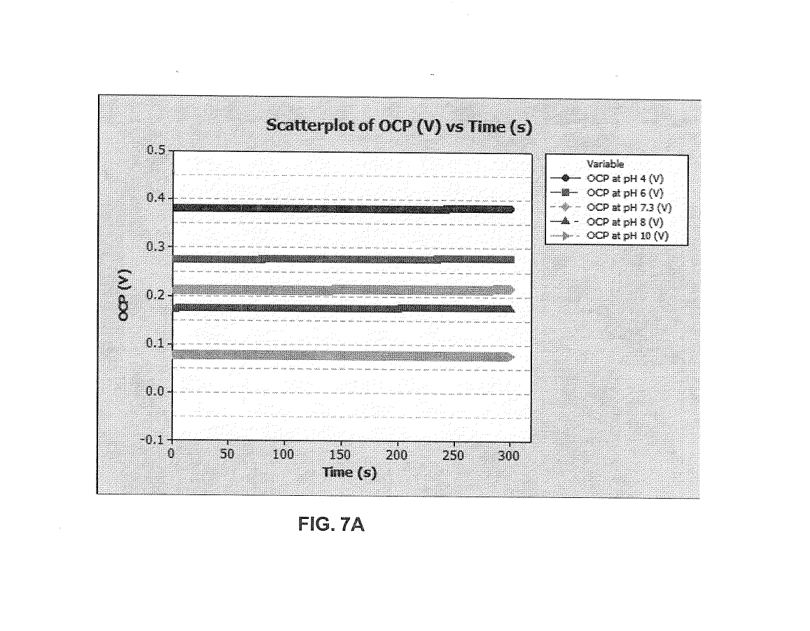

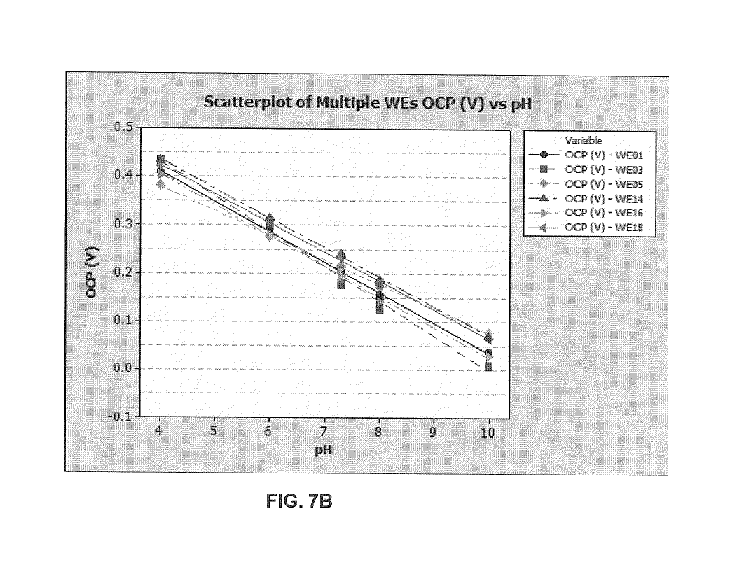

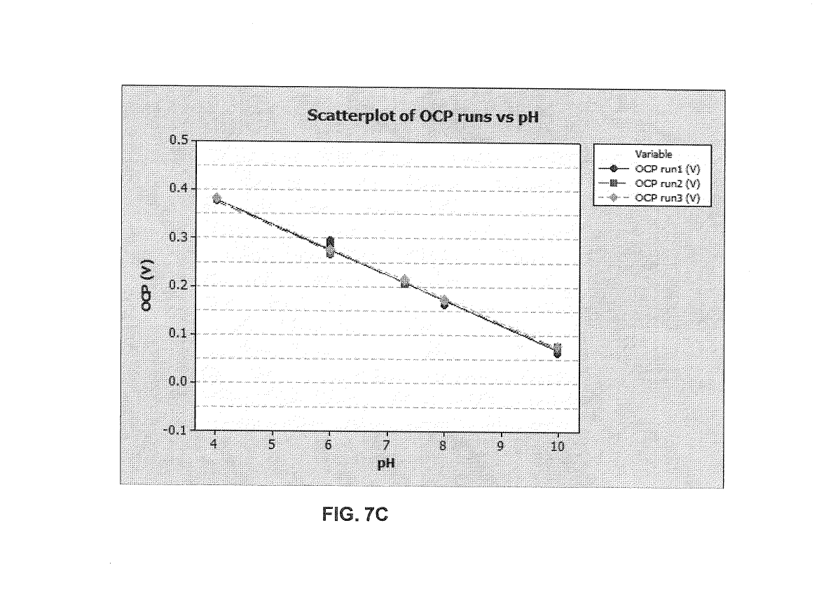

FIG. 2 provides a perspective view of one generalized embodiment of subcutaneous sensor insertion system and a block diagram of a sensor electronics device according to one illustrative embodiment of the invention. Additional elements typically used with such sensor system embodiments are disclosed for example in U.S. Patent Application No. 20070163894, the contents of which are incorporated by reference. FIG. 2 provides a perspective view of a telemetered characteristic monitor system 1, including a subcutaneous sensor set 10 provided for subcutaneous placement of an active portion of a flexible sensor 12, or the like, at a selected site in the body of a user. The subcutaneous or percutaneous portion of the sensor set 10 includes a hollow, slotted insertion needle 14 having a sharpened tip 44, and a cannula 16. Inside the cannula 16 is a sensing portion 18 of the sensor 12 to expose one or more sensor electrodes 20 to the user's bodily fluids through a window 22 formed in the cannula 16. The sensing portion 18 is joined to a connection portion 24 that terminates in conductive contact pads, or the like, which are also exposed through one of the insulative layers. The connection portion 24 and the contact pads are generally adapted for a direct wired electrical connection to a suitable monitor 200 coupled to a display 214 for monitoring a user's condition in response to signals derived from the sensor electrodes 20. The connection portion 24 may be conveniently connected electrically to the monitor 200 or a characteristic monitor transmitter 100 by a connector block 28 (or the like).

As shown in FIG. 2, in accordance with embodiments of the present invention, subcutaneous sensor set 10 may be configured or formed to work with either a wired or a wireless characteristic monitor system. The proximal part of the sensor 12 is mounted in a mounting base 30 adapted for placement onto the skin of a user. The mounting base 30 can be a pad having an underside surface coated with a suitable pressure sensitive adhesive layer 32, with a peel-off paper strip 34 normally provided to cover and protect the adhesive layer 32, until the sensor set 10 is ready for use. The mounting base 30 includes upper and lower layers 36 and 38, with the connection portion 24 of the flexible sensor 12 being sandwiched between the layers 36 and 38. The connection portion 24 has a forward section joined to the active sensing portion 18 of the sensor 12, which is folded angularly to extend downwardly through a bore 40 formed in the lower base layer 38. Optionally, the adhesive layer 32 (or another portion of the apparatus in contact with in vivo tissue) includes an anti-inflammatory agent to reduce an inflammatory response and/or anti-bacterial agent to reduce the chance of infection. The insertion needle 14 is adapted for slide-fit reception through a needle port 42 formed in the upper base layer 36 and through the lower bore 40 in the lower base layer 38. After insertion, the insertion needle 14 is withdrawn to leave the cannula 16 with the sensing portion 18 and the sensor electrodes 20 in place at the selected insertion site. In this embodiment, the telemetered characteristic monitor transmitter 100 is coupled to a sensor set 10 by a cable 102 through a connector 104 that is electrically coupled to the connector block 28 of the connector portion 24 of the sensor set 10.

In the embodiment shown in FIG. 2, the telemetered characteristic monitor 100 includes a housing 106 that supports a printed circuit board 108, batteries 110, antenna 112, and the cable 102 with the connector 104. In some embodiments, the housing 106 is formed from an upper case 114 and a lower case 116 that are sealed with an ultrasonic weld to form a waterproof (or resistant) seal to permit cleaning by immersion (or swabbing) with water, cleaners, alcohol or the like. In some embodiments, the upper and lower case 114 and 116 are formed from a medical grade plastic. However, in alternative embodiments, the upper case 114 and lower case 116 may be connected together by other methods, such as snap fits, sealing rings, RTV (silicone sealant) and bonded together, or the like, or formed from other materials, such as metal, composites, ceramics, or the like. In other embodiments, the separate case can be eliminated and the assembly is simply potted in epoxy or other moldable materials that is compatible with the electronics and reasonably moisture resistant. As shown, the lower case 116 may have an underside surface coated with a suitable pressure sensitive adhesive layer 118, with a peel-off paper strip 120 normally provided to cover and protect the adhesive layer 118, until the sensor set telemetered characteristic monitor transmitter 100 is ready for use.

In the illustrative embodiment shown in FIG. 2, the subcutaneous sensor set 10 facilitates accurate placement of a flexible thin film electrochemical sensor 12 of the type used for monitoring specific blood parameters representative of a user's condition. The sensor 12 monitors glucose levels in the body, and may be used in conjunction with automated or semi-automated medication infusion pumps of the external or implantable type as described in U.S. Pat. No. 4,562,751; 4,678,408; 4,685,903 or 4,573,994, to control delivery of insulin to a diabetic patient.

In the illustrative embodiment shown in FIG. 2, the sensor electrodes 10 may be used in a variety of sensing applications and may be configured in a variety of ways. For example, the sensor electrodes 10 may be used in physiological parameter sensing applications in which some type of biomolecule is used as a catalytic agent. For example, the sensor electrodes 10 may be used in a glucose and oxygen sensor having a glucose oxidase enzyme catalyzing a reaction with the sensor electrodes 20. The sensor electrodes 10, along with a biomolecule or some other catalytic agent, may be placed in a human body in a vascular or non-vascular environment. For example, the sensor electrodes 20 and biomolecule may be placed in a vein and be subjected to a blood stream, or may be placed in a subcutaneous or peritoneal region of the human body.

In the embodiment of the invention shown in FIG. 2, the monitor of sensor signals 200 may also be referred to as a sensor electronics device 200. The monitor 200 may include a power source, a sensor interface, processing electronics (i.e. a processor), and data formatting electronics. The monitor 200 may be coupled to the sensor set 10 by a cable 102 through a connector that is electrically coupled to the connector block 28 of the connection portion 24. In an alternative embodiment, the cable may be omitted. In this embodiment of the invention, the monitor 200 may include an appropriate connector for direct connection to the connection portion 104 of the sensor set 10. The sensor set 10 may be modified to have the connector portion 104 positioned at a different location, e.g., on top of the sensor set to facilitate placement of the monitor 200 over the sensor set.

While the analyte sensor and sensor systems disclosed herein are typically designed to be implantable within the body of a mammal, the inventions disclosed herein are not limited to any particular environment and can instead be used in a wide variety of contexts, for example for the analysis of most in vivo and in vitro liquid samples including biological fluids such as interstitial fluids, whole-blood, lymph, plasma, serum, saliva, urine, stool, perspiration, mucus, tears, cerebrospinal fluid, nasal secretion, cervical or vaginal secretion, semen, pleural fluid, amniotic fluid, peritoneal fluid, middle ear fluid, joint fluid, gastric aspirate or the like. In addition, solid or desiccated samples may be dissolved in an appropriate solvent to provide a liquid mixture suitable for analysis.

It is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. In the description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

1. An amperometric analyte sensor system comprising:
a base;
a plurality of electrodes disposed on the base including:
a working electrode;
a counter electrode;
a reference electrode;
a pH electrode responsive to changes in pH within the sensor system;
a processor; and
a computer-readable program having instructions which cause the processor to assess signal data obtained from the working electrode and the pH electrode; wherein:
the working electrode and the processor are coupled so that the working electrode monitors analyte within the sensor system;
the pH electrode and the processor are coupled so that the pH electrode monitors pH within the sensor system; and
the processor uses a first algorithm to calculate a concentration of analyte when the pH of the sensor system is at or above pH 7.1; and
the processor uses a second algorithm to calculate a concentration of analyte when the pH of the sensor system is below pH 6.9.

2. The amperometric analyte sensor system of claim 1, wherein the second algorithm calculates the concentration of analyte considering an at least 10% drop in analyte signal that results from the pH of the sensor system changing from at or above pH 7.1 to below pH 6.9.

3. The amperometric analyte sensor system of claim 1, wherein the working electrode is coated with a plurality of layered materials comprising:
an analyte sensing layer comprising an oxidoreductase that produces an acidic compound in the presence of analyte;
an interference rejection layer;
a protein layer;
an adhesion promoting layer; and/or
an analyte modulating layer, wherein the analyte modulating layer comprises a composition that modulates the diffusion of an analyte diffusing through the analyte modulating layer.

4. The amperometric analyte sensor system of claim 1, wherein the working electrode comprises platinum black coated with a glucose oxidase composition that forms gluconic acid and hydrogen peroxide in the presence of glucose.

5. The amperometric analyte sensor system of claim 1, wherein the pH electrode comprises a metal, a metal oxide, a polymer and/or a hydrogel.

6. The amperometric analyte sensor system of claim 1, wherein the pH electrode and the working electrode are both in operable contact with the reference electrode and the counter electrode.

7. The amperometric analyte sensor system of claim 1, wherein the pH electrode continuously monitors the open circuit potential between the pH electrode and the reference electrode.

8. The amperometric analyte sensor system of claim 7, wherein the first and second algorithms include a determination of how pH modulates amperometric current observed at the working electrode in the presence of analyte.

9. The amperometric analyte sensor system of claim 1, wherein the pH electrode functions as the working electrode.

10. A method of calculating the concentration of glucose at a plurality of different pH values within an amperometric glucose sensor, the method comprising:
(a) placing an amperometric glucose sensor into an environment comprising glucose, where the amperometric analyte sensor is disposed within a system comprising:
a base;
a plurality of electrodes disposed on the base including:
a working electrode, wherein the working electrode is coated with:
an analyte sensing layer comprising glucose oxidase that produces gluconic acid and hydrogen peroxide in the presence of glucose; and
an analyte modulating layer, wherein the analyte modulating layer comprises a composition that modulates the diffusion of an analyte diffusing through the analyte modulating layer;
a counter electrode;
a reference electrode;
a pH electrode responsive to changes in pH within the local sensor system environment;
a processor; and
a computer-readable program having instructions which cause the processor to:
assess signal data obtained from the working electrode and the pH electrode; wherein:
the working electrode and the processor are coupled so that the working electrode monitors glucose within the sensor system;
the pH electrode and the processor are coupled so that the pH electrode monitors the pH of the sensor within the sensor system;
(b) monitoring the pH of the sensor within the sensor system;
(c) monitoring glucose within the sensor system; and
(d) calculating the concentration of glucose, wherein:
the processor uses a first set of parameters to calculate a concentration of glucose when the pH of the analyte sensing layer is at or above pH 7.1; and
the processor uses a second set of parameters to calculate a concentration of glucose when the pH of the analyte sensing layer is below pH 6.9.

11. The method of claim 10, wherein the second set of parameters calculates the concentration of analyte using an at least 10% drop in analyte signal that results from the pH of the sensor system changing from at or above pH 7.1 to below pH 6.9.

12. The method of claim 10, wherein the pH electrode continuously monitors the open circuit potential between the pH electrode and the reference electrode.

13. The method of claim 12, wherein the system switches from using the first set of parameters to using the second set of parameters when the open circuit potential is above or below a predefined value that is between 20 millivolts and 180 millivolts.

14. The method of claim 10, wherein the method includes using a calibration curve of the relationship between current and pH at the working electrode within the sensor.

15. A method of making an analyte sensor comprising the steps of:
providing a base layer;
forming a conductive layer on the base layer, wherein the conductive layer includes a plurality of electrodes including a pH electrode, a working electrode, a reference electrode and a counter electrode;
forming an analyte sensing layer over the working electrode, wherein the analyte sensing layer comprises a polypeptide that forms an acidic compound in the presence of the analyte; and
forming an analyte modulating layer disposed over the analyte sensing layer, wherein the analyte modulating layer includes a composition that modulates the diffusion of the analyte therethrough.
forming an adhesion promoting layer on the analyte sensing layer or the protein layer; or
forming a cover layer disposed on at least a portion of the analyte modulating layer, wherein the cover layer further includes an aperture over at least a portion of the analyte modulating layer.

16. The method of claim 15, wherein the analyte modulating layer comprises:
(1) a polyurethane/polyurea polymer formed from a mixture comprising:
(a) a diisocyanate;
(b) a hydrophilic polymer comprising a hydrophilic diol or hydrophilic diamine; and
(c) a siloxane having an amino, hydroxyl or carboxylic acid functional group at a terminus; and/or
(2) a branched acrylate polymer formed from a mixture comprising:
(a) a butyl, propyl, ethyl or methyl-acrylate;
(b) an amino-acrylate;
(c) a siloxane-acrylate; and
(d) a poly(ethylene oxide)-acrylate.

17. The method of claim 15, wherein the analyte sensor apparatus operably coupled to a process comprising a computer-readable program having instructions which cause the processor to assess signal data obtained from the working electrode and the pH electrode; wherein:
the pH electrode and the processor are coupled so that the pH electrode monitors pH of the sensor within the sensor system; and
the processor uses a first algorithm to calculate a concentration of analyte when the pH of the sensor system is at or above pH 7.1; and
the processor uses a second algorithm to calculate a concentration of analyte when the pH of the sensor system is below pH 6.9.

18. The method of claim 15, wherein the pH electrode is adapted to continuously monitor open circuit potential between the pH electrode and the reference electrode.

19. The method of claim 18, wherein the system switches from using the first algorithm to using the algorithm when the open circuit potential is above or below a predefined value that is between 20 millivolts and 180 millivolts.

20. The method of claim 15, wherein a single electrode functions the pH electrode and the working electrode.
